(19) United States
(12) Patent Application Publication
(10) Pub. No.: US 2013/0052665 A1
(43) Pub. Date: Feb. 28, 2013

(54) METHODS FOR DIAGNOSIS OF SYSTEMIC JUVENILE IDIOPATHIC ARTHRITIS

(71) Applicants: Bruce Xuefeng Ling, Palo Alto, CA (US); Elizabeth D. Mellins, Stanford, CA (US)

(72) Inventors: Bruce Xuefeng Ling, Palo Alto, CA (US); Elizabeth D. Mellins, Stanford, CA (US)

(21) Appl. No.: 13/589,727

(22) Filed: Aug. 20, 2012

Related U.S. Application Data
(60) Provisional application No. 61/527,533, filed on Aug. 25, 2011.

(51) Int. Cl. G01N 33/566; G01N 21/64

(52) U.S. Cl. 435/7.92; 436/501

(57) ABSTRACT

Methods for diagnosis of systemic juvenile idiopathic arthritis (SJIA) are disclosed. In particular, the invention relates to the use of biomarkers for diagnosis of SJIA, which can be used to distinguish SJIA from other inflammatory diseases, including infectious illness, acute febrile illness, Kawasaki disease, and similar juvenile idiopathic arthritis (JIA) disease subtypes, and to predict inflammatory flares in SJIA patients in advance of clinical symptoms.

### CROSS-REFERENCE TO RELATED APPLICATION

This application claims benefit under 35 U.S.C. §119(e) of provisional application 61/527,533, filed Aug. 25, 2011, which application is hereby incorporated by reference in its entirety.

### STATEMENT REGARDING FEDERALLY SPONSORED RESEARCH OR DEVELOPMENT

This invention was made with Government support under contracts AI075254 and AR050942 awarded by the National Institutes of Health. The Government has certain rights in this invention.

### TECHNICAL FIELD

The present invention pertains generally to methods for diagnosis of systemic juvenile idiopathic arthritis (SJIA). In particular, the invention relates to the use of biomarkers for diagnosis of SJIA, which can be used to distinguish SJIA from other inflammatory diseases, including infectious illness, acute febrile illness, Kawasaki disease, and similar juvenile idiopathic arthritis (JIA) disease subtypes, and to predict inflammatory flares in SJIA patients.

### BACKGROUND

Systemic juvenile idiopathic arthritis (SJIA) is a chronic disease in children characterized by a combination of arthritis and systemic inflammation. The course of the disease is typically persistent or polycyclic with periods of disease flare and quiescence in about 60% of affected subjects (Singh-Grewal et al. (2006) Arthritis Rheum. 54 1595-1601; Schneider et al. (1998) Baillieres Clin. Rheumatol. 12:245-1271). Approximately 40% of patients experience a monocyclic course of disease that resolves. Upon diagnosis, patients usually present with nonspecific evidence of inflammation, such as an elevated erythrocyte sedimentation rate (ESR). However, there is currently no specific diagnostic test, and it can be difficult to discriminate SJIA from other inflammatory illness, such as Kawasaki disease, infectious illness, and febrile illness. Thus, there remains a need for sensitive and specific diagnostic tests for SJIA that can discriminate SJIA from other inflammatory conditions.

### SUMMARY

The invention relates to the use of biomarkers for diagnosis of SJIA. In particular, the inventors have discovered a panel of biomarkers whose expression profile can be used to diagnose SJIA and to distinguish SJIA from other inflammatory diseases, including infectious illness, acute febrile illness, Kawasaki disease, and other Juvenile Idiopathic Arthritis (JIA) disease subtypes. The inventors have further shown that this panel of biomarkers can be used to predict incipient inflammatory flares in SJIA patients in advance of clinical symptoms.

In one aspect, the invention includes a method for diagnosing SJIA in a subject. The method comprises (i) measuring the level of a plurality of biomarkers in a biological sample derived from a subject; and (ii) analyzing the levels of the biomarkers and comparing with respective reference value ranges for the biomarkers, wherein differential expression of one or more biomarkers in the biological sample compared to one or more biomarkers in a control sample obtained from a healthy individual, who does not have SJIA, indicates that the subject has SJIA.

In certain embodiments, the level of one or more biomarkers is compared with reference value ranges for the biomarkers. The reference value ranges can represent the level of one or more biomarkers found in one or more samples of one or more subjects without SJIA (i.e., normal samples). Alternatively, the reference values can represent the level of one or more biomarkers found in one or more samples of one or more subjects with SJIA.

Biomarkers that can be used in the practice of the invention include, but are not limited to, alpha-1-antichymotrypsin (ACT), alpha-1-acid glycoprotein (AGP1), alpha-2-macroglobulin (A2M), inter-alpha-trypsin inhibitor light chain (AMBP), apolipoprotein A1 (APO A-I), apolipoprotein A-IV (APO A-IV), apolipoprotein D (APO D), apolipoprotein E (APO E), apolipoprotein L1 (APO L1), antithrombin III (ATIII), complement C3 (C3), complement C4 (C4), complement C9 (C9), C-reactive protein (CRP), fibrinogen β (FGB), fibrinogen γ (FGG), gelsolin (GSN), complement factor H (CFH), haptoglobin (HP), kininogenin (KLKB1), calgranulin A (S100A8/MRP8), calgranulin B (S100A9/MRP14), serum amyloid A (SAA), serum amyloid P (SAP), transthyretin (TTR), and vitamin D binding protein (VDB). In one embodiment, a panel of biomarkers comprising A2M, APO-AI, CRP, HP, S100A8/S100A9, SAA, and SAP is used for diagnosis of SJIA. In certain embodiments, the panel of biomarkers further comprises one or more biomarkers selected from the group consisting of transthyretin (TTR), calgranulin C (S100A12), complement factor H (CFH), gelsolin (GSN), complement C4 (C4), alpha-1-acid glycoprotein (AGP1), alpha-1-antichymotrypsin (ACT), and apolipoprotein A-IV (APO A-IV).

Biomarkers can be measured by performing an enzyme-linked immunosorbent assay (ELISA), a radioimmunoassay (RIA), an immunofluorescent assay (IFA), a sandwich assay, magnetic capture, microsphere capture, a Western Blot, surface enhanced Raman spectroscopy (SERS), flow cytometry, or mass spectrometry. In certain embodiments, the level of a biomarker is measured by contacting an antibody with the biomarker, wherein the antibody specifically binds to the biomarker, or a fragment thereof containing an antigenic determinant of the biomarker. Antibodies that can be used in the practice of the invention include, but are not limited to, monoclonal antibodies, polyclonal antibodies, chimeric antibodies, recombinant fragments of antibodies, Fab fragments, Fab′ fragments, F(ab′)₂ fragments, Fᵥ fragments, or scFᵥ fragments.

Methods of the invention, as described herein, can be used to distinguish a diagnosis of SJIA for a subject from infectious illness, acute febrile illness, Kawasaki disease, or polyarticular JIA. In one embodiment, the invention includes a method to determine whether the subject is in a state of SJIA disease flare or disease quiescence. Methods of the invention can be used to further predict incipient SJIA disease flares up to 9 weeks in advance of clinical flare symptoms in a subject.

In certain embodiments, a panel of biomarkers is used for diagnosis of SJIA. Biomarker panels of any size can be used in the practice of the invention. Biomarker panels for diagnosing SJIA typically comprise at least 4 biomarkers and up to 30 biomarkers, including any number of biomarkers in between, such as 4, 5, 6, 7, 8, 9, 10, 11, 12, 13, 14, 15, 16, 17, 18, 19, 20, 21, 22, 23, 24, 25, 26, 27, 28, 29, or 30 biomarkers. For example, the biomarker panel may comprise 2-4 biomarkers, 5-7 biomarkers, 8-10 biomarkers, 10-15, biomarkers, 15-20 biomarkers, 20-25 biomarkers, or 25-30 biomarkers. In certain embodiments, the invention includes a biomarker panel comprising at least 4, or at least 5, or at least 6, or at least 7, or at least 8, or at least 9, or at least 10 or more biomarkers. Although smaller biomarker panels are usually more economical, larger biomarker panels (i.e., greater than 30 biomarkers) have the advantage of providing more detailed information and can also be used in the practice of the invention.

In one embodiment, the invention includes a biomarker panel comprising A2M, APO-AI, CRP, HP, S100A8/S100A9, SAA, and SAP. In certain embodiments, the biomarker panel further comprises one or more biomarkers selected from the group consisting of transthyretin (TTR), calgranulin C (S100A12), complement factor H (CFH), gelsolin (GSN), complement C4 (C4), alpha-1-acid glycoprotein (AGP1), alpha-1-antichymotrypsin (ACT), and apolipoprotein A-IV (APO A-IV).

In another embodiment, the invention includes a method for evaluating the effect of an agent for treating SJIA in a subject, the method comprising: analyzing the level of each of one or more SJIA biomarkers in biological samples derived from the subject before and after the subject is treated with the agent, and comparing the levels of the biomarkers with respective reference value ranges for the biomarkers.

In another embodiment, the invention includes a method for monitoring the efficacy of a therapy for treating SJIA in a subject, the method comprising: analyzing the level of each of one or more SJIA biomarkers in biological samples derived from the subject before and after the subject undergoes the therapy, and comparing the levels of the biomarkers with respective reference value ranges for the biomarkers.

In another aspect, the invention includes a kit for diagnosing SJIA in a subject. The kit may include at least one agent for detecting an SJIA biomarker, a container for holding a biological sample isolated from a human subject suspected of having SJIA, and printed instructions for reacting the agent with the biological sample or a portion of the biological sample to detect the presence or amount of at least one SJIA biomarker in the biological sample. The agents may be packaged in separate containers. The kit may further comprise one or more control reference samples and reagents for performing an immunoassay. In one embodiment, the kit comprises agents for measuring the levels of at least seven biomarkers of interest, including A2M, APO-AI, CRP, HP, S100A8/S100A9, SAA, and SAP. The kit may include antibodies that specifically bind to these biomarkers, for example, the kit may contain at least one of an antibody that specifically binds to A2M, an antibody that specifically binds to APO-AI, an antibody that specifically binds to CRP, an antibody that specifically binds to HP, an antibody that specifically binds to S100A8/S100A9, an antibody that specifically binds to SAA, and an antibody that specifically binds to SAP.

These and other embodiments of the subject invention will readily occur to those of skill in the art in view of the disclosure herein.

### BRIEF DESCRIPTION OF THE FIGURES

FIG. 1 shows a heatmap display of unsupervised hierarchical clustering of the relative protein abundance (normalized volume data, low-light gray and high-dark gray) in paired SJIA F/Q plasma samples. The rows of heatmap represent the 89 gel spots derived from 26 different proteins (labeled with SwissProt protein names at the left of the heatmap) with each column of that row representing a different sample from subjects with SJIA flare (black) and SJIA quiescence (light gray). The SJIA F sample, clustered with the SJIA Q branch, is labeled with a dark gray star.

FIG. 2 shows a heatmap display of the unsupervised clustering analyses of all detected ATIII, C4, C9, SAA and A2M protein spots. Estimated molecular weight (MW) and isoelectric point (pI) are indicated. The wrongly clustered SJIA F sample (FIG. 1) is labeled with a dark gray star in the heatmap of each protein.

FIGS. 3A and 3B show the construction of a robust SJIA flare panel. FIG. 3A shows a false discovery rate (FDR) analysis of the 26 proteins discriminating SJIA F and Q. X-Y plot of FDR as a function of the number of proteins called significant. FIG. 3B shows a heatmap display of unsupervised clustering analyses of expression of the top 15 proteins, ranked by the nearest shrunken centroid algorithm (NSC), in SJIA F/Q, Poly JIA F/Q, SJIA F/KD, SJIA F/FI samples. The misclassified SJIA F sample (FIG. 1) is labeled with a dark gray star in each heatmap.

FIGS. 4A and 4B shows the selection of the 7 ELISA biomarker panel and validation of DIGE results. FIG. 4A shows the goodness of separation analysis to select the optimal biomarker panel size for SJIA flare ELISA analysis. Using ELISA data from SJIA F/Q training and test data sets, as indicated, various classifiers of different panel size (feature #) were tested for their goodness of separation between flare (dark gray) and quiescence (light gray) as shown by the box-whisker graphs. Boxes contain the 50% of values falling between the 25th and 75th percentiles; the horizontal line within the box represents the median value and the “whisker” lines extend to the highest and lowest values. FIG. 4B shows ELISA assays validating biomarker observations from DIGE assays. The box-whisker graphs illustrate the spread of the protein abundance of each biomarker from SJIA F/Q, KD and FI samples using either DIGE or ELISA assays. Boxes contain the 50% of values falling between the 25th and 75^{th} percentiles; the horizontal line within the box represents the median value and the “whisker” lines extend to the highest and lowest values.

FIGS. 5A-5C show a linear discriminant analysis of the ELISA-based SJIA flare biomarker panel differentiating SJIA F from Q samples. FIG. 5A shows the SJIA flare biomarker panel of 7 ELISA assays. Linear discriminant analysis (LDA) was performed with training data from SJIA F (n=17) and Q (n=17) samples evaluated with the biomarker panel. Estimated probabilities for the training (left) and test data (right) are plotted. Samples are partitioned by the true class (upper) and predicted class (lower). The maximum estimated probability for each of the wrongly assigned samples is marked with a dark gray arrow. The trained LDA model was tested using an independent data set from SJIA F (n=10) and Q (n=10) samples. FIG. 5B shows the classification results from training and test sets are shown as 2×2 contingency tables. Fisher exact test was used to measure P values of the 2×2 tables with (upper) and without (lower) confounding F samples. FIG. 5C shows ROC analyses, using training, test or combined training and testing data sets, to compare the SJIA F and Q classification performance by either ESR, S100A8/S100A9, CRP or SJIA flare ELISA panel, respectively.

FIGS. 6A and 6B show a linear discriminant analysis of the 7-protein SJIA flare biomarker panel, differentiating SJIA F from FI subjects. FIG. 6A shows the LDA analysis. SJIA F (n=22) and FI (n=27) subjects were used to develop a binary-class classifier. Samples are partitioned by the true class (upper) and predicted class (lower). The maximum estimated probability for each of the wrongly assigned samples is marked with a dark gray arrow. The LDA classification results are shown as a 2×2 contingency table. Fisher exact test was used to measure the statistical significance (P value) of the 2×2 table. FIG. 6B shows ROC analyses of the effectiveness of the biomarker panel to discriminate SJIA F from FI, which was compared to either S100A8/S100A9, CRP or ESR, respectively.

FIGS. 7A and 7B show a linear discriminant analysis of the ELISA-based SJIA flare biomarker panel in detection of impending SJIA flare. QF: 10 SJIA quiescent samples drawn within 2-9 weeks of a clinical flare; QQ: 10 SJIA quiescent controls who remained in quiescence for 6 months after the sample was drawn. FIG. 7A shows estimated probabilities for the training (left) and test data (right). Samples are partitioned by the true class (upper) and predicted class (lower). The maximum estimated probability for each of the wrongly assigned samples is marked with a dark gray arrow. SJIA QQ and QF samples were used as training set to develop a binary classifier. The classification results are shown as a 2×2 contingency table, comparing SJIA QF to QQ. The Fisher exact test was used to measure the P value of the 2×2 table. FIG. 7B shows ROC analyses, using training data sets to compare the SJIA F and Q classification performance by ESR, S100A8/S100A9, CRP or SJIA flare panel.

FIG. 8 shows a pathway analysis of the proteins in the SJIA signature. Data mining software (Ingenuity Systems, ingenuity.com, CA) was used with differentially (F vs. Q) expressed plasma proteins to identify gene ontology groups and relevant canonical signaling pathways associated with SJIA flare. The intensity of the node color indicates the degree of up- (dark gray) or down- (light gray) regulation in SJIA F. Nodes are displayed using shapes that represent the functional class of the gene product and different relationships are represented by line type (see key). Relationships are primarily due to co-expression, but can also include phosphorylation/dephosphorylation, proteolysis, activation/deactivation, transcription, binding, inhibition, biochemical modification.

FIGS. 9A and 9B show an analysis of the protein profiles differentiating SJIA F from KD subjects. FIG. 9A shows a heatmap display of unsupervised clustering analyses of expression of the top 9 proteins with Student's t test P value<0.05 comparing SJIA F and KD samples. The miss-clustered SJIA F sample (shown in FIG. 1 and FIG. 3 labeled with a dark gray star) by the SJIA F panel when comparing SJIA F to either SJIA Q or FI is also miss-clustered when comparing SJIA F and KD (labeled with a dark gray star). FIG. 9B shows the analysis with data mining software (Ingenuity Systems, ingenuity.com, CA), which was used with differentially (SJIA F vs. KD) expressed plasma proteins to identify gene ontology groups and relevant canonical signaling pathways associated with SJIA flare. The intensity of the node color indicates the degree of up- (dark gray) or down- (light gray) regulation in SJIA F. Nodes are displayed using shapes that represent the functional class of the gene product and different relationships are represented by line type (see key). Relationships are primarily due to coexpression, but can also include phosphorylation/dephosphorylation, proteolysis, activation/deactivation, transcription, binding, inhibition, biochemical modification.

FIG. 10 shows a composite gray scale image view of the 2-D Difference Gel Electrophoresis (DIGE), including all protein species from SJIA flare, SJIA quiescent, Poly JIA flare, Poly JIA quiescent, Kawasaki Disease and febrile illness control samples. A total of 89 spots (labeled by arrows) from 26 different protein precursors (right panel) were identified by mass spectrometric analysis. Different species of the same protein with different molecular weights (MW) and isoelectric points (pI) were labeled with the same index number but different alphabetical labels.

FIG. 11 shows ELISA assays (CRP, HP, SAA and S100A8/14) of SJIA F and Q samples from three centers (Stanford University, UCSD and UCSF). The protein abundance in each sample was initially measured as μg/ml, normalized to the total protein amount (mg), and then scaled using the scale function from R base package. The box-whisker graphs illustrate the spread of the protein abundance for each biomarker from either F or Q samples from the indicated center. Boxes contain the 50% of values falling between the 25th and 75th percentiles; the horizontal line within the box represents the median value and the “whisker” lines extend to the highest and lowest values.

FIGS. 12A-12C shows a linear discriminant analysis of the 7-protein SJIA flare biomarker panel applied to Poly JIA F and Q samples. FIG. 12A shows that Poly JIA F (n=13) and Q (n=10) samples were used as a training set to develop a predictive model, based on LDA. The trained LDA model was tested using an independent data set with Poly JIA F (n=10) and Q (n=5) subjects. Estimated probabilities for the training (left) and test data (right) are shown. Samples are partitioned by the true class (upper) and predicted class (lower). The maximum estimated probability for each of the wrongly assigned samples is marked with a gray arrow. FIG. 12B shows the classification results from both training (A) and test (B) are shown as a 2×2 contingency table. The Fisher exact test was used to compute the P value. FIG. 12C shows the results of ROC analyses using training and test data sets, which were performed to compare the Poly JIA F and Q classification performance by either CRP or SJIA flare panel, respectively.

### DETAILED DESCRIPTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a biomarker” includes a mixture of two or more biomarkers, and the like.

The term “about”, particularly in reference to a given quantity, is meant to encompass deviations of plus or minus five percent.

A “biomarker” in the context of the present invention refers to a biological compound, which is differentially expressed in a sample taken from patients having SJIA as compared to a comparable sample taken from control subjects (e.g., a person with a negative diagnosis, normal or healthy subject). The biomarker can be a protein, a fragment of a protein, a peptide, or a polypeptide. SJIA biomarkers include, but are not limited to, alpha-1-antichymotrypsin (ACT), alpha-1-acid glycoprotein (AGP1), alpha-2-macroglobulin (A2M), inter-alpha-trypsin inhibitor light chain (AMBP), apolipoprotein A1 (APO A-I), apolipoprotein A-IV (APO A-IV), apolipoprotein D (APO D), apolipoprotein E (APO E), apolipoprotein L1 (APO L1), antithrombin III (ATIII), complement C3 (C3), complement C4 (C4), complement C9 (C9), C-reactive protein (CRP), fibrinogen β(FGB), fibrinogen γ (FGG), gelsolin (GSN), complement factor H-related protein 1 (H36), haptoglobin (HP), kininogenin (KLKB1), calgranulin A (S100A8/MRP8), calgranulin B (S100A9/MRP14), serum amyloid A (SAA), serum amyloid P (SAP), transthyretin (TTR), vitamin D binding protein (VDB), and fragments thereof, or variants thereof comprising amino acid sequences displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto.

The terms “polypeptide” and “protein” refer to a polymer of amino acid residues and are not limited to a minimum length. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation, hydroxylation, oxidation, and the like.

The phrase “differentially expressed” refers to differences in the quantity and/or the frequency of a biomarker present in a sample taken from patients having, for example, SJIA as compared to a control subject. For example, a biomarker can be a polypeptide which is present at an elevated level or at a decreased level in samples of patients with SJIA compared to samples of control subjects. Alternatively, a biomarker can be a polypeptide which is detected at a higher frequency or at a lower frequency in samples of patients compared to samples of control subjects. A biomarker can be differentially present in terms of quantity, frequency or both.

A polypeptide is differentially expressed between two samples if the amount of the polypeptide in one sample is statistically significantly different from the amount of the polypeptide in the other sample. For example, a polypeptide is differentially expressed in two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially expressed in two sets of samples if the frequency of detecting the polypeptide in samples of patients' suffering from SJIA, is statistically significantly higher or lower than in the control samples. For example, a polypeptide is differentially expressed in two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; and primates.

As used herein, a “biological sample” refers to a sample of tissue or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies and also samples of in vitro cell culture constituents, including but not limited to, conditioned media resulting from the growth of cells and tissues in culture medium, e.g., recombinant cells, and cell components.

A “test amount” of a marker refers to an amount of a biomarker present in a sample being tested. A test amount can be either an absolute amount (e.g., μg/ml) or a relative amount (e.g., relative intensity of signals).

A “diagnostic amount” of a biomarker refers to an amount of a biomarker in a subject's sample that is consistent with a diagnosis of SJIA. A diagnostic amount can be either an absolute amount (e.g., μg/ml) or a relative amount (e.g., relative intensity of signals).

A “control amount” of a marker can be any amount or a range of amount which is to be compared against a test amount of a marker. For example, a control amount of a biomarker can be the amount of a biomarker in a person without SJIA. A control amount can be either in absolute amount (e.g., μg/ml) or a relative amount (e.g., relative intensity of signals).

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

“Immunoassay” is an assay that uses an antibody to specifically bind an antigen (e.g., a biomarker). The immunoassay is characterized by the use of specific binding properties of a particular antibody to isolate, target, and/or quantify the antigen. An immunoassay for a biomarker may utilize one antibody or several antibodies. Immunoassay protocols may be based, for example, upon competition, direct reaction, or sandwich type assays using, for example, labeled antibody. The labels may be, for example, fluorescent, chemiluminescent, or radioactive.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a biomarker from specific species such as rat, mouse, or human can be selected to obtain only those polyclonal antibodies that are specifically immunoreactive with the biomarker and not with other proteins, except for polymorphic variants and alleles of the biomarker. This selection may be achieved by subtracting out antibodies that cross-react with biomarker molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Capture reagent” refers to a molecule or group of molecules that specifically bind to a specific target molecule or group of target molecules. For example, a capture reagent can comprise two or more antibodies each antibody having specificity for a separate target molecule. Capture reagents can be any combination of organic or inorganic chemicals, or biomolecules, and all fragments, analogs, homologs, conjugates, and derivatives thereof that can specifically bind a target molecule.

The capture reagent can comprise a single molecule that can form a complex with multiple targets, for example, a multimeric fusion protein with multiple binding sites for different targets. The capture reagent can comprise multiple molecules each having specificity for a different target, thereby resulting in multiple capture reagent-target complexes. In certain embodiments, the capture reagent is comprised of proteins, such as antibodies.

The capture reagent can be directly labeled with a detectable moiety. For example, an anti-biomarker antibody can be directly conjugated to a detectable moiety and used in the inventive methods, devices, and kits. In the alternative, detection of the capture reagent-biomarker complex can be by a secondary reagent that specifically binds to the biomarker or the capture reagent-biomarker complex. The secondary reagent can be any biomolecule, and is preferably an antibody. The secondary reagent is labeled with a detectable moiety. In some embodiments, the capture reagent or secondary reagent is coupled to biotin, and contacted with avidin or streptavidin having a detectable moiety tag.

“Detectable moieties” or “detectable labels” contemplated for use in the invention include, but are not limited to, radioisotopes, fluorescent dyes such as fluorescein, phycoerythrin, Cy-3, Cy-5, allophycoyanin, DAPI, Texas Red, rhodamine, Oregon green, Lucifer yellow, and the like, green fluorescent protein (GFP), red fluorescent protein (DsRed), Cyan Fluorescent Protein (CFP), Yellow Fluorescent Protein (YFP), Cerianthus Orange Fluorescent Protein (cOFP), alkaline phosphatase (AP), beta-lactamase, chloramphenicol acetyltransferase (CAT), adenosine deaminase (ADA), aminoglycoside phosphotransferase (neo^{r}, G418^{r}) dihydrofolate reductase (DHFR), hygromycin-B-phosphotransferase (HPH), thymidine kinase (TK), lacZ (encoding α-galactosidase), and xanthine guanine phosphoribosyltransferase (XGPRT), Beta-Glucuronidase (gus), Placental Alkaline Phosphatase (PLAP), Secreted Embryonic Alkaline Phosphatase (SEAP), or Firefly or Bacterial Luciferase (LUC). Enzyme tags are used with their cognate substrate. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), and glass microparticles with digital holographic code images (see e.g., CyVera microbeads produced by Illumina (San Diego, Calif.). As with many of the standard procedures associated with the practice of the invention, skilled artisans will be aware of additional labels that can be used.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

“Substantially purified” refers to nucleic acid molecules or proteins that are removed from their natural environment and are isolated or separated, and are at least about 60% free, preferably about 75% free, and most preferably about 90% free, from other components with which they are naturally associated.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of biomarkers that can be used in the diagnosis of SJIA. In particular, the inventors have discovered a panel of biomarkers whose expression profile can be used to diagnose SJIA and to distinguish SJIA from other inflammatory diseases, including infectious illness, acute febrile illness, Kawasaki disease, and other Juvenile Idiopathic Arthritis (JIA) disease subtypes (see Example 1). The inventors have further shown that this panel of biomarkers can be used to predict incipient inflammatory flares in SJIA patients up to 9 weeks in advance of clinical symptoms (see Example 1). In order to further an understanding of the invention, a more detailed discussion is provided below regarding the identified biomarkers and methods of using them in diagnosis of SJIA.

A. Biomarkers

Biomarkers that can be used in the practice of the invention include, but are not limited to, alpha-1-antichymotrypsin (ACT), alpha-1-acid glycoprotein (AGP1), alpha-2-macroglobulin (A2M), inter-alpha-trypsin inhibitor light chain (AMBP), apolipoprotein A1 (APO A-I), apolipoprotein A-IV (APO A-IV), apolipoprotein D (APO D), apolipoprotein E (APO E), apolipoprotein L1 (APO L1), antithrombin III (ATIII), complement C3 (C3), complement C4 (C4), complement C9 (C9), C-reactive protein (CRP), fibrinogen β (FGB), fibrinogen γ (FGG), gelsolin (GSN), complement factor H (CFH), haptoglobin (HP), kininogenin (KLKB1), calgranulin A (S100A8/MRP8), calgranulin B (S100A9/MRP14), serum amyloid A (SAA), serum amyloid P (SAP), transthyretin (TTR), and vitamin D binding protein (VDB), and fragments thereof, or variants thereof comprising amino acid sequences displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto. In one embodiment, a panel of biomarkers comprising A2M, APO-AI, CRP, HP, S100A8/S100A9, SAA, and SAP is used for diagnosis of SJIA. In certain embodiments, the panel of biomarkers further comprises one or more biomarkers selected from the group consisting of transthyretin (TTR), calgranulin C (S100A12), complement factor H (CFH), gelsolin (GSN), complement C4 (C4), alpha-1-acid glycoprotein (AGP1), alpha-1-antichymotrypsin (ACT), and apolipoprotein A-IV (APO A-IV). Differential expression of these biomarkers is associated with SJIA, and therefore expression profiles of these biomarkers are useful for diagnosing SJIA and distinguishing SJIA disease from other inflammatory conditions, including infectious illness, acute febrile illness, Kawasaki disease, and other Juvenile Idiopathic Arthritis (JIA) disease subtypes.

Accordingly, in one aspect, the invention provides a method for diagnosing SJIA in a subject, comprising measuring the level of a plurality of biomarkers in a biological sample derived from a subject suspected of having SJIA, and analyzing the levels of the biomarkers and comparing with respective reference value ranges for the biomarkers, wherein differential expression of one or more biomarkers in the biological sample compared to one or more biomarkers in a control sample indicates that the subject has SJIA. When analyzing the levels of biomarkers in a biological sample, the reference value ranges used for comparison can represent the level of one or more biomarkers found in one or more samples of one or more subjects without SJIA (i.e., normal or control samples). Alternatively, the reference values can represent the level of one or more biomarkers found in one or more samples of one or more subjects with SJIA.

The biological sample obtained from the subject to be diagnosed is typically blood or plasma, but can be any sample from bodily fluids, tissue or cells that contain the expressed biomarkers. A “control” sample as used herein refers to a biological sample, such as blood, plasma, tissue, or cells that are not diseased. That is, a control sample is obtained from a normal subject (e.g. an individual known to not have SJIA or any condition or symptom associated with the disease). A biological sample can be obtained from a subject by conventional techniques. For example, blood can be obtained by venipuncture, while plasma and serum can be obtained by fractionating whole blood according to known methods. Surgical techniques for obtaining solid tissue samples are well known in the art.

In certain embodiments, a panel of biomarkers is used for diagnosis of SJIA. Biomarker panels of any size can be used in the practice of the invention. Biomarker panels for diagnosing SJIA typically comprise at least 4 biomarkers and up to 30 biomarkers, including any number of biomarkers in between, such as 4, 5, 6, 7, 8, 9, 10, 11, 12, 13, 14, 15, 16, 17, 18, 19, 20, 21, 22, 23, 24, 25, 26, 27, 28, 29, or 30 biomarkers. For example, the biomarker panel may comprise 2-4 biomarkers, 5-7 biomarkers, 8-10 biomarkers, 10-15, biomarkers, 15-20 biomarkers, 20-25 biomarkers, or 25-30 biomarkers. In certain embodiments, the invention includes a biomarker panel comprising at least 4, or at least 5, or at least 6, or at least 7, or at least 8, or at least 9, or at least 10 or more biomarkers. Although smaller biomarker panels are usually more economical, larger biomarker panels (i.e., greater than 30 biomarkers) have the advantage of providing more detailed information and can also be used in the practice of the invention.

In certain embodiments, the biomarker panel comprises 7 or more biomarkers for diagnosing SJIA. In one embodiment, the invention includes a biomarker panel comprising A2M, APO-AI, CRP, HP, S100A8/S100A9, SAA, and SAP. In certain embodiments, the biomarker panel further comprises one or more biomarkers selected from the group consisting of transthyretin (TTR), calgranulin C (S100A12), complement factor H (CFH), gelsolin (GSN), complement C4 (C4), alpha-1-acid glycoprotein (AGP1), alpha-1-antichymotrypsin (ACT), and apolipoprotein A-IV (APO A-IV). Biomarkers panels are useful for diagnosing SJIA and distinguishing SJIA disease from other inflammatory conditions, including infectious illness, acute febrile illness, Kawasaki disease, and other Juvenile Idiopathic Arthritis (JIA) disease subtypes. Biomarker panels are also useful for predicting inflammatory flares in SJIA patients up to 9 weeks in advance of clinical symptoms (see Example 1).

B. Detecting and Measuring Levels of Biomarkers

It is understood that the expression level of the biomarkers in a sample can be determined by any suitable method known in the art. Measurement of the expression level of a biomarker can be direct or indirect. For example, the abundance levels of RNAs or proteins can be directly quantitated. Alternatively, the amount of a biomarker can be determined indirectly by measuring abundance levels of cDNAs, amplified RNAs or DNAs, or by measuring quantities or activities of RNAs, proteins, or other molecules (e.g., metabolites) that are indicative of the expression level of the biomarker.

In one embodiment, the expression levels of the biomarkers are determined by measuring levels of proteins or polypeptide or peptide fragments thereof. Assays based on the use of antibodies that specifically recognize the proteins or polypeptide or peptide fragments of the biomarkers may be used for the measurement. Such assays include, but are not limited to, immunohistochemistry (IHC), western blotting, enzyme-linked immunosorbent assay (ELISA), radioimmunoassays (RIA), “sandwich” immunoassays, fluorescent immunoassays, immunoprecipitation assays, the procedures of which are well known in the art (see, e.g., Ausubel et al, eds, 1994, Current Protocols in Molecular Biology, Vol. 1, John Wiley & Sons, Inc., New York, which is incorporated by reference herein in its entirety). Antibodies that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). A biomarker antigen can be used to immunize a mammal, such as a mouse, rat, rabbit, guinea pig, monkey, or human, to produce polyclonal antibodies. If desired, a biomarker antigen can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a biomarker antigen can be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256, 495-97, 1985; Kozbor et al., J. Immunol. Methods 81, 3142, 1985; Cote et al., Proc. Natl. Acad. Sci. 80, 2026-30, 1983; Cole et al., Mol. Cell. Biol. 62, 109-20, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-55, 1984; Neuberger et al., Nature 312, 604-08, 1984; Takeda et al., Nature 314, 452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332. Human monoclonal antibodies can be prepared in vitro as described in Simmons et al., PLoS Medicine 4(5), 928-36, 2007.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., Eur. J. Cancer Prev. 5, 507-11, 1996). Single-chain antibodies can be mono- or bispecific, and can be bivalent or tetravalent. Construction of tetravalent, bispecific single-chain antibodies is taught, for example, in Coloma & Morrison, Nat. Biotechnol. 15, 159-63, 1997. Construction of bivalent, bispecific single-chain antibodies is taught in Mallender & Voss, J. Biol. Chem. 269, 199-206, 1994.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., Int. J. Cancer 61, 497-501, 1995; Nicholls et al., J. Immunol. Meth. 165, 81-91, 1993).

Antibodies which specifically bind to a biomarker antigen also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies can be purified by methods well known in the art. For example, antibodies can be affinity purified by passage over a column to which the relevant antigen is bound. The bound antibodies can then be eluted from the column using a buffer with a high salt concentration.

Antibodies may be used in diagnostic assays to detect the presence or for quantification of the biomarkers in a biological sample. Such a diagnostic assay may comprise at least two steps; (i) contacting a biological sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278(2):123-131 (2000)), or a chromatography column, etc; and (ii) quantifying the antibody bound to the substrate. The method may additionally involve a preliminary step of attaching the antibody, either covalently, electrostatically, or reversibly, to a solid support, before subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as ²H, ¹⁴C, ³²P, or ¹²⁵I, a fluorescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem., 13:1014 (1974); Pain et al., J. Immunol. Methods, 40:219 (1981); and Nygren, J. Histochem. and Cytochem., 30:407 (1982).

Immunoassays can be used to determine the presence or absence of a biomarker in a sample as well as the quantity of a biomarker in a sample. First, a test amount of a biomarker in a sample can be detected using the immunoassay methods described above. If a biomarker is present in the sample, it will form an antibody-biomarker complex with an antibody that specifically binds the biomarker under suitable incubation conditions, as described above. The amount of an antibody-biomarker complex can be determined by comparing to a standard. A standard can be, e.g., a known compound or another protein known to be present in a sample. As noted above, the test amount of a biomarker need not be measured in absolute units, as long as the unit of measurement can be compared to a control.

It may be useful in the practice of the invention to fractionate biological samples, e.g., to enrich samples for lower abundance plasma proteins to facilitate detection of biomarkers, or to partially purify biomarkers isolated from biological samples to generate specific antibodies to biomarkers. There are many ways to reduce the complexity of a sample based on the binding properties of the proteins in the sample, or the characteristics of the proteins in the sample.

In one embodiment, a sample can be fractionated according to the size of the proteins in a sample using size exclusion chromatography. For a biological sample wherein the amount of sample available is small, preferably a size selection spin column is used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by immunoassays, gas phase ion spectrometry, and the like, for the detection of biomarkers.

In another embodiment, a sample can be fractionated by anion exchange chromatography. Anion exchange chromatography allows fractionation of the proteins in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of biomarkers in a sample that are more negatively charged from other types of biomarkers. Proteins that are eluted with an eluant having a high pH are likely to be weakly negatively charged, and proteins that are eluted with an eluant having a low pH are likely to be strongly negatively charged. Thus, in addition to reducing complexity of a sample, anion exchange chromatography separates proteins according to their binding characteristics.

In yet another embodiment, a sample can be fractionated by heparin chromatography. Heparin chromatography allows fractionation of the biomarkers in a sample also on the basis of affinity interaction with heparin and charge characteristics. Heparin, a sulfated mucopolysaccharide, will bind biomarkers with positively charged moieties, and a sample can be sequentially eluted with eluants having different pH's or salt concentrations. Biomarkers eluted with an eluant having a low pH are more likely to be weakly positively charged. Biomarkers eluted with an eluant having a high pH are more likely to be strongly positively charged. Thus, heparin chromatography also reduces the complexity of a sample and separates biomarkers according to their binding characteristics.

In yet another embodiment, a sample can be fractionated by isolating proteins that have a specific characteristic, e.g. glycosylation. For example, a sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

In yet another embodiment, a sample can be fractionated using a sequential extraction protocol. In sequential extraction, a sample is exposed to a series of adsorbents to extract different types of biomarkers from a sample. For example, a sample is applied to a first adsorbent to extract certain proteins, and an eluant containing non-adsorbent proteins (i.e., proteins that did not bind to the first adsorbent) is collected. Then, the fraction is exposed to a second adsorbent. This further extracts various proteins from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomarkers. In this embodiment, the sample is applied to a first adsorbent on the probe, which is subsequently washed with an eluant. Biomarkers that do not bind to the first adsorbent are removed with an eluant. The biomarkers that are in the fraction can be applied to a second adsorbent on the probe, and so forth. The advantage of performing sequential extraction on a gas phase ion spectrometer probe is that biomarkers that bind to various adsorbents at every stage of the sequential extraction protocol can be analyzed directly using a gas phase ion spectrometer.

In yet another embodiment, biomarkers in a sample can be separated by high-resolution electrophoresis, e.g., one or two-dimensional gel electrophoresis. A fraction containing a biomarker can be isolated and further analyzed by gas phase ion spectrometry. Preferably, two-dimensional gel electrophoresis is used to generate a two-dimensional array of spots for the biomarkers. See, e.g., Jungblut and Thiede, Mass Spectr. Rev. 16:145-162 (1997).

Two-dimensional gel electrophoresis can be performed using methods known in the art. See, e.g., Deutscher ed., Methods In Enzymology vol. 182. Typically, biomarkers in a sample are separated by, e.g., isoelectric focusing, during which biomarkers in a sample are separated in a pH gradient until they reach a spot where their net charge is zero (i.e., isoelectric point). This first separation step results in one-dimensional array of biomarkers. The biomarkers in the one dimensional array are further separated using a technique generally distinct from that used in the first separation step. For example, in the second dimension, biomarkers separated by isoelectric focusing are further resolved using a polyacrylamide gel by electrophoresis in the presence of sodium dodecyl sulfate (SDS-PAGE). SDS-PAGE allows further separation based on molecular mass. Typically, two-dimensional gel electrophoresis can separate chemically different biomarkers with molecular masses in the range from 1000-200,000 Da, even within complex mixtures.

Biomarkers in the two-dimensional array can be detected using any suitable methods known in the art. For example, biomarkers in a gel can be labeled or stained (e.g., Coomassie Blue or silver staining). If gel electrophoresis generates spots that correspond to the molecular weight of one or more biomarkers of the invention, the spot can be further analyzed by densitometric analysis or gas phase ion spectrometry. For example, spots can be excised from the gel and analyzed by gas phase ion spectrometry. Alternatively, the gel containing biomarkers can be transferred to an inert membrane by applying an electric field. Then a spot on the membrane that approximately corresponds to the molecular weight of a biomarker can be analyzed by gas phase ion spectrometry. In gas phase ion spectrometry, the spots can be analyzed using any suitable techniques, such as MALDI or SELDI.

Prior to gas phase ion spectrometry analysis, it may be desirable to cleave biomarkers in the spot into smaller fragments using cleaving reagents, such as proteases (e.g., trypsin). The digestion of biomarkers into small fragments provides a mass fingerprint of the biomarkers in the spot, which can be used to determine the identity of the biomarkers if desired.

In yet another embodiment, high performance liquid chromatography (HPLC) can be used to separate a mixture of biomarkers in a sample based on their different physical properties, such as polarity, charge and size. HPLC instruments typically consist of a reservoir, the mobile phase, a pump, an injector, a separation column, and a detector. Biomarkers in a sample are separated by injecting an aliquot of the sample onto the column. Different biomarkers in the mixture pass through the column at different rates due to differences in their partitioning behavior between the mobile liquid phase and the stationary phase. A fraction that corresponds to the molecular weight and/or physical properties of one or more biomarkers can be collected. The fraction can then be analyzed by gas phase ion spectrometry to detect biomarkers.

Optionally, a biomarker can be modified before analysis to improve its resolution or to determine its identity. For example, the biomarkers may be subject to proteolytic digestion before analysis. Any protease can be used. Proteases, such as trypsin, that are likely to cleave the biomarkers into a discrete number of fragments are particularly useful. The fragments that result from digestion function as a fingerprint for the biomarkers, thereby enabling their detection indirectly. This is particularly useful where there are biomarkers with similar molecular masses that might be confused for the biomarker in question. Also, proteolytic fragmentation is useful for high molecular weight biomarkers because smaller biomarkers are more easily resolved by mass spectrometry. In another example, biomarkers can be modified to improve detection resolution. For instance, neuraminidase can be used to remove terminal sialic acid residues from glycoproteins to improve binding to an anionic adsorbent and to improve detection resolution. In another example, the biomarkers can be modified by the attachment of a tag of particular molecular weight that specifically binds to molecular biomarkers, further distinguishing them. Optionally, after detecting such modified biomarkers, the identity of the biomarkers can be further determined by matching the physical and chemical characteristics of the modified biomarkers in a protein database (e.g., SwissProt).

After preparation, biomarkers in a sample are typically captured on a substrate for detection. Traditional substrates include antibody-coated 96-well plates or nitrocellulose membranes that are subsequently probed for the presence of the proteins. Alternatively, protein-binding molecules attached to microspheres, microparticles, microbeads, beads, or other particles can be used for capture and detection of biomarkers. The protein-binding molecules may be antibodies, peptides, peptoids, aptamers, small molecule ligands or other protein-binding capture agents attached to the surface of particles. Each protein-binding molecule may comprise a “unique detectable label,” which is uniquely coded such that it may be distinguished from other detectable labels attached to other protein-binding molecules to allow detection of biomarkers in multiplex assays. Examples include, but are not limited to, color-coded microspheres with known fluorescent light intensities (see e.g., microspheres with xMAP technology produced by Luminex (Austin, Tex.); microspheres containing quantum dot nanocrystals, for example, having different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.); chemiluminescent dyes, combinations of dye compounds; and beads of detectably different sizes. See, e.g., U.S. Pat. No. 5,981,180, U.S. Pat. No. 7,445,844, U.S. Pat. No. 6,524,793, Rusling et al. (2010) Analyst 135(10): 2496-2511; Kingsmore (2006) Nat. Rev. Drug Discov. 5(4): 310-320, Proceedings Vol. 5705 Nanobiophotonics and Biomedical Applications II, Alexander N. Cartwright; Marek Osinski, Editors, pp. 114-122; Nanobiotechnology Protocols Methods in Molecular Biology, 2005, Volume 303; herein incorporated by reference in their entireties).

In another example, biochips can be used for capture and detection of proteins. Many protein biochips are described in the art. These include, for example, protein biochips produced by Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture reagent bound there. The capture reagent can be a biological molecule, such as a polypeptide or a nucleic acid, which captures other biomarkers in a specific manner. Alternatively, the capture reagent can be a chromatographic material, such as an anion exchange material or a hydrophilic material. Examples of such protein biochips are described in the following patents or patent applications: U.S. Pat. No. 6,225,047 (Hutchens and Yip, “Use of retentate chromatography to generate difference maps,” May 1, 2001), International publication WO 99/51773 (Kuimelis and Wagner, “Addressable protein arrays,” Oct. 14, 1999), International publication WO 00/04389 (Wagner et al., “Arrays of protein-capture agents and methods of use thereof,” Jul. 27, 2000), International publication WO 00/56934 (Englert et al., “Continuous porous matrix arrays,” Sep. 28, 2000).

In general, a sample containing the biomarkers is placed on the active surface of a biochip for a sufficient time to allow binding. Then, unbound molecules are washed from the surface using a suitable eluant. In general, the more stringent the eluant, the more tightly the proteins must be bound to be retained after the wash. The retained protein biomarkers now can be detected by any appropriate means, for example, mass spectrometry, fluorescence, surface plasmon resonance, ellipsometry or atomic force microscopy.

Mass spectrometry, and particularly SELDI mass spectrometry, is a particularly useful method for detection of the biomarkers of this invention. Laser desorption time-of-flight mass spectrometer can be used in embodiments of the invention. In laser desorption mass spectrometry, a substrate or a probe comprising biomarkers is introduced into an inlet system. The biomarkers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry (MALDI-MS) can also be used for detecting the biomarkers of this invention. MALDI-MS is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS, the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry.

Surface-enhanced laser desorption/ionization mass spectrometry, or SELDI-MS represents an improvement over MALDI for the fractionation and detection of biomolecules, such as proteins, in complex mixtures. SELDI is a method of mass spectrometry in which biomolecules, such as proteins, are captured on the surface of a protein biochip using capture reagents that are bound there. Typically, non-bound molecules are washed from the probe surface before interrogation. SELDI is described, for example, in: U.S. Pat. No. 5,719,060 (“Method and Apparatus for Desorption and Ionization of Analytes,” Hutchens and Yip, Feb. 17, 1998) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichesher, 2000.

Biomarkers on the substrate surface can be desorbed and ionized using gas phase ion spectrometry. Any suitable gas phase ion spectrometer can be used as long as it allows biomarkers on the substrate to be resolved. Preferably, gas phase ion spectrometers allow quantitation of biomarkers. In one embodiment, a gas phase ion spectrometer is a mass spectrometer. In a typical mass spectrometer, a substrate or a probe comprising biomarkers on its surface is introduced into an inlet system of the mass spectrometer. The biomarkers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of biomarkers or other substances will typically involve detection of signal intensity. This, in turn, can reflect the quantity and character of biomarkers bound to the substrate. Any of the components of a mass spectrometer (e.g., a desorption source, a mass analyzer, a detector, etc.) can be combined with other suitable components described herein or others known in the art in embodiments of the invention.

The methods for detecting biomarkers in a sample have many applications. For example, one or more biomarkers can be measured to aid in the diagnosis of SJIA. In another example, the methods for detection of the biomarkers can be used to monitor responses in a subject to treatment. In another example, the methods for detecting biomarkers can be used to assay for and to identify compounds that modulate expression of these biomarkers in vivo or in vitro.

C. Kits

In yet another aspect, the invention provides kits for diagnosing SJIA, wherein the kits can be used to detect the biomarkers of the present invention. For example, the kits can be used to detect any one or more of the biomarkers described herein, which are differentially expressed in samples of an SJIA patient and normal subjects. The kit may include one or more agents for detection of biomarkers, a container for holding a biological sample isolated from a human subject suspected of having SJIA; and printed instructions for reacting agents with the biological sample or a portion of the biological sample to detect the presence or amount of at least one SJIA biomarker in the biological sample. The agents may be packaged in separate containers. The kit may further comprise one or more control reference samples and reagents for performing an immunoassay.

In one embodiment, the kit comprises agents for measuring the levels of at least seven biomarkers of interest, including A2M, APO-AI, CRP, HP, S100A8/S100A9, SAA, and SAP. The kit may include antibodies that specifically bind to these biomarkers, for example, the kit may contain at least one of an antibody that specifically binds to A2M, an antibody that specifically binds to APO-AI, an antibody that specifically binds to CRP, an antibody that specifically binds to HP, an antibody that specifically binds to S100A8/S100A9, an antibody that specifically binds to SAA, and an antibody that specifically binds to SAP.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of diagnosing SJIA.

The kits of the invention have a number of applications. For example, the kits can be used to determine if a subject has SJIA or some other inflammatory condition arising, for example, from infectious illness, acute febrile illness, or Kawasaki disease, and to distinguish a diagnosis of SJIA from another juvenile idiopathic arthritis (JIA) disease subtype. In another example, the kits can be used to predict incipient SJIA inflammatory flares in advance of clinical symptoms in a subject. In another example, kits can be used to monitor the effectiveness of treatment of a patient having SJIA. In a further example, the kits can be used to identify compounds that modulate expression of one or more of the biomarkers in in vitro or in vivo animal models to determine the effects of treatment.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Identifying Biomarkers for Systemic Juvenile Idiopathic Arthritis (SJIA)

### Study Subjects

Children with SJIA and poly JIA were recruited from the Pediatric Rheumatology Clinics at Lucile Packard Children's Hospital, Stanford, Calif., USA from 2000 to 2008 and at the University of California, San Francisco (UCSF) from 2006 to 2008. All children with juvenile arthritis were recruited on the basis of the American College of Rheumatology (ACR) criteria; all were identified retrospectively to have met the 1997 International League of Associations for Rheumatology (ILAR) criteria for JIA (Petty et al. (2004) International League of Associations for Rheumatology classification of juvenile idiopathic arthritis: second revision, Edmonton, 2001. The Journal of Rheumatology 31:390-392). Serial peripheral blood samples were collected from these subjects, including samples from periods of active disease noted as flare (F) and inactive disease noted as quiescence (Q). Comprehensive clinical data were also collected from these subjects. We studied 10 SJIA subjects with paired F and Q samples by 2D-DIGE. For the ELISA analysis, we used matched F/Q samples from 18 SJIA subjects (9/18 subjects also provided samples for the 2D-DIGE analysis) and samples from 4 additional subjects at F and 4 additional (unmatched) subjects at Q. As serial samples were available on most subjects, there was only 1 SJIA F sample and 5 SJIA Q samples used in both ELISA and DIGE studies. To construct training and testing F and Q cohorts for ELISA analyses, all samples were randomized with the consideration that similar portions of the samples are matched F/Q in training and testing sets (5 subjects, 10/24, 41.2% samples and 4 subjects, 8/20, 40% samples, respectively). 15 SJIA subjects contributed Q samples in the prediction of flare ELISA experiment; no samples in this analysis were used in any other assay. We also studied 5 poly JIA subjects with paired F and Q samples by 2D-DIGE. For the ELISA analysis, we used matched F/Q samples from 15 poly JIA subjects (2/15 also provided samples for the 2D-DIGE analysis) and samples from 8 additional poly JIA subjects at F. There were matched F/Q samples from 2 Poly JIA subjects and 1 Poly JIA F sample (unmatched) used in both ELISA and DIGE studies. Clinical and demographic characteristics of JIA subjects included in the study are summarized in Tables 1A/B, 2A/B and 3.

For KD and FI samples, subjects who presented to the Emergency Department at Rady Children's Hospital San Diego and met study criteria were enrolled from 2004 to 2008. Inclusion criteria for children with KD were 4 out of 5 standard clinical criteria (rash, conjunctival injection, cervical lymphadenopathy, changes in the extremities, changes in the oropharynx) or 3 of 5 criteria with dilated coronary arteries by echocardiogram. All KD patient samples were taken prior to intravenous immunoglobulin (IVIG) treatment. Inclusion criteria for the other febrile children were fever for at least 3 days accompanied by any of the following signs: rash; conjunctival injection; cervical lymphadenopathy; oropharyngeal erythema; or peripheral edema. Enrolled subjects were ultimately found to have the following diagnoses: SJIA, scarlet fever, viral syndrome, staphylococcal abscess (methicillin-resistant and methicillin-sensitive), streptococcal adenitis, bacterial urinary tract infection, viral meningitis, perirectal abscess, and Henoch Schonlein purpura (HSP). Clinical and demographic characteristics of KD and FI subjects included in the study are summarized in Tables 1B and 2B. Each subject provided a single blood sample at study enrollment. There was no overlap between the KD and FI subjects/samples studied by DIGE and those studied by ELISA.

Protocols for these studies were approved by the institutional review boards at the clinical centers, and all parents gave written consent for the participation of their child. Child and adolescent assent were obtained as appropriate.

### Clinical Variables and Scoring System

Comprehensive clinical and clinical laboratory data on children with JIA were collected in association with each plasma sample. To facilitate correlation between clinical information and proteomic data, we developed a scoring system to grade severity of systemic disease manifestations and arthritis (Supplemental Table 1A/B/C). Scores were assigned by a pediatric rheumatologist, who reviewed the medical record, including clinical laboratory data. The systemic scoring (Supplemental Table 1A) is based on the results of hierarchical clustering analysis of SJIA subjects with early (<3 months) active disease (Sandborg et al. (2006) Journal of Rheumatology 33:2322-2329). Arthritis scoring (Supplemental Tables 1B, 1C) is based on the number of “active” joints, defined as swelling or limitation of motion with pain in an affected joint. The scoring of arthritis severity is different for SJIA and poly JIA subjects, because the patterns of joint involvement are different between the 2 groups (Weiss et al. (2005) Pediatr. Clin. North Am. 52:413-442; Schneider et al. (1998) Baillieres Clin. Rheumatol. 12:245-271). The scoring is based on differences in frequency analysis of numbers of active joints in early active SJIA compared to active poly JIA (Sandborg et al. (2006) Journal of Rheumatology 33:2322-2329 and C. Sandborg, unpublished data). For the purpose of this study, we defined a flare sample as one with a systemic score greater than 0 and an arthritis score>A (SJIA subjects) or >0 (poly JIA subjects). A quiescent sample was defined as one with a systemic score of 0 or an arthritis score of A (SJIA) or 0 (poly JIA). 9 of 10 SJIA F samples used in our 2D-DIGE analysis were from subjects with both active arthritis and systemic disease activity; the remaining subject had only active systemic features without clinically detectable arthritis. All SJIA F samples used in our ELISA validation studies were from subjects with both active arthritis and systemic disease activity.

### Plasma Preparation

For samples obtained at Stanford and UCSF, venous blood was collected in EDTA, heparin or citrate when blood was drawn for clinical laboratory determination of complete blood count, differential and erythrocyte sedimentation rate. Within 2 hours of draw, whole blood samples were centrifuged at 25° C. at 514 g for 5 minutes to remove cells and spun an additional two times at 4° C. at 1730 g for 5 and 15 minutes respectively to remove platelets. Processed plasma samples were stored at −80° C. until analysis. No findings reported in this study could be attributed to differences in anticoagulant used (data not shown). Blood samples from SJIA, KD, and FI at UCSD were collected in EDTA and centrifuged within one hour to isolate plasma, which was stored at −80° C. until analysis.

### Sample Preparation and Protein Labeling

To enrich samples for lower abundance plasma proteins, plasma samples were depleted of six abundant proteins (albumin, IgA, IgG, haptoglobin, transferrin, and alpha-1 antitrypsin) using Agilent Multiple Affinity Removal System (Agilent, Santa Clara, Calif.). Specifically, the depletion enabled the increased loading of the remaining proteins by ten-fold (not shown). Depleted plasma was precipitated to separate proteins from detergents, salts, lipids, phenolics, and nucleic acids using a 2-D clean-up kit (GE Healthcare Biosciences, Pittsburgh, Pa.). Protease tablets (Roche Applied Science, Branford, Conn.) were dissolved as per manufacturer's instructions and applied as part of the buffer during the sample processing and 2-D clean-up processes. Protein concentrations were subsequently measured by protein microassay, using bovine serum albumin as a standard (Bio-Rad, Hercules, Calif.). Equal amounts of protein from paired F and Q samples were tagged with Cy3 or Cy5, respectively. Pooled standards were labeled with Cy2 and consisted of equal amounts of protein from all samples in the experiment. A 1 mM CyDye stock solution with dimethylformamide (DMF) was used.

### Two Dimensional Gel Electrophoresis (2D DIGE)

Dye-labeled plasma protein samples were mixed with rehydration buffer (7 M urea, 2 M thiourea, 4% CHAPS, 0.0001% bromophenol blue) and pipetted across pH 3-10 NL BioRad Ready Strip IPG strips (Bio-Rad) inside a rehydration strip tray. Mineral oil was overlayed across each strip and the strip was rehydrated for 48 hours, after which protein samples were focused using Protean IEF focusing equipment at 200 volts for 10 hours, 500 volts for 2 hours, 1,000 volts for 2 hours, 5,000 volts for 2 hours, and 10,000 volts for 8 hours at 20° C.

Isoelectrofocused IPG strips were placed in SDS equilibration buffer (6 M urea; 50 mM Tris, pH 8.8; 30% glycerol, 2% SDS, 0.0002% bromophenol blue) for 15 minutes. IPG strips were subsequently loaded onto the second dimension gels, which contained 10% acrylamide, 375 mM Tris-C1, pH 8.8, 0.05% APS, and 0.05% TEMED and were sealed by 1% agarose containing 2% SDS and 50 mM Tris, pH 6.8. The second dimension of protein electrophoresis was performed using an ETTAN DALT vertical system with a current of 2 watts per gel for 16 hours at 10° C.

### 2D-DIGE Analysis

Gels were imaged on the Typhoon 9400 Variable Mode Imager at specific wavelengths that excited Cy2, Cy3, or Cy5, allowing the protein profile of a given sample to be measured as soon as the second dimension was completed. Quantitative analysis was done on every spot on each gel using Progenesis software from Non-Linear USA Inc. (Durham, N.C.). Gel images went through a process of image quality assessment and were aligned to create protein spot index. We performed three sets of 2D-DIGE experiments: matched-pair F and Q from SJIA subjects (n=10), matched-pair F and Q from polyJIA subjects (n=5), and 12 KD/12 FI subjects with the pooled standards from each of the two previous experiments. The third experiment served as the bridge, allowing protein gel spot alignment, normalization and indexing across all assayed samples. Molecular weight and pI calibration were performed with Progenesis software using a set of protein spots with known MW and pI as internal standards.

### Protein Identification via MALDI-TOF/TOF Mass Spectrometry

Differentially expressed protein spots were excised from the gel using an Ettan Spot Picker (GE Healthcare Biosciences), digested with trypsin, spotted to a MALDI target and analyzed in MS and MS/MS modes with an Applied Biosystems 4700 MALDI-TOF/TOF mass spectrometer. Mass spectrometry data were analyzed by Mascot (Matrix Science Inc., Boston, Mass.) to identify proteins from primary sequence databases. An experienced mass spectroscopist reviewed all identification data before acceptance. Criteria for a positive identification were a statistically significant score for the peptide mass fingerprint (MS data) and amino acid sequence for two peptides (MS/MS data).

### ELISA Assays

ELISA assays were performed to measure plasma levels of selected proteins: serum amyloid A (SAA), S100A8, S100A9, S100A8/S100A9 complex (calprotectin), serum amyloid P (SAP), Creactive protein (CRP), haptoglobin (HP), apolipoprotein A-1 (APO AI), alpha-2-macroglobulin (A2M) and S100A12. All assays, except for SAP and S1000A12, were performed using commercial ELISA kits: A2M, plasma was diluted 1:400 and assayed by AssayMax (St. Charles, Mo.) kit; APO A1, plasma was diluted 1:800 and assayed by AssayMax kit; S100A8 and S100A9, plasma was diluted 1:100 and assayed by kits from BMA Biomedicals (Augst, Switzerland); calprotectin, plasma was diluted 1:100 and assayed by Cell Sciences (Canton, Mass.) kit; CRP, plasma was diluted 1:250 and assayed by Immunology Consultants Laboratory (Newberg, Oreg.) kit; HP, plasma was diluted 1:50,000 and assayed by Immunology Consultants Laboratory kit; SAA, plasma was diluted 1:50 and assayed by Antigenix America (Huntington Station, N.Y.) kit; For SAP analysis, ELISA plates (MaxiSorp; Nunc, Rochester, N.Y.) were coated with SAP antibody (Millipore, Billerica, Mass.) with PBS overnight at 4° C. at 10 μg/ml. Plates were blocked with 1% bovine serum albumin (BSA) in phosphate buffered saline (PBS) (Sigma, St. Louis, Mo.) for 1 hr at 37° C. Plates were washed and incubated with sample (diluted 1:1000) for 1 hour at room temperature (RT) followed by another wash and subsequent incubation with anti-SAP rabbit antibody (Abcam, Cambridge, Mass.) diluted 1:100 with PBS plus 10% FBS, pH 7.0 for 1 hour at 37° C. Anti-rabbit horseradish peroxidase (HRP) (Southern Biotech, Birmingham, Ala.) diluted to 1:4000 was added for 30 minutes at 37° C. For determination of S100A12, ELISA plates were coated with S100A12 antibody (Abcam) in carbonate buffer overnight at 4° C. at 1 μg/well. Plates were blocked with 0.25% BSA in PBS (Sigma) for 1 hr at 37° C. Plates were washed and incubated with sample (diluted 1:3) for 2 hours at room temperature followed by another wash and subsequent incubation with biotin-conjugated anti-S100A12 antibody (R&D Systems, Minneapolis, Minn.) at 0.25 μg/well for 30 minutes at 37° C. Streptavidin conjugated to horseradish peroxidase (HRP) (Invitrogen, Carlsbad, Calif.) at 0.8 μg/ml in PBS was added for 30 minutes at 37° C. Substrate buffer followed by stop solution was added, and absorbance was read at 405 nm.

### Statistical Analyses

Patient demographic data was analyzed using “Epidemiological calculator” (R epicalc package). Hypothesis testing used Student t test and Mann-Whitney U test, and local FDR (Efron et al. (2001) Journal of the American Statistical Association 96:1151-1160) to correct for multiple hypothesis testing issues. Nearest shrunken centroid (NSC) based feature selection, including permutation based FDR analysis, was performed using R PAM package (Tibshirani et al. (2002) Proceedings of the National Academy of Sciences of the United States of America 99:6567-6572). Unsupervised heatmap analyses were performed using R stats package. Binary class clustering results were grouped into modified 2×2 contingency tables, which were used to calculate the proportion of the clustering results that agreed with clinical diagnosis and the statistical significance by the Fisher's exact test. Supervised linear discriminant analysis for binary (SJIA F vs. Q, Poly JIA F vs. Q) and three class (SJIA F, KD and FI) classifications, using R MASS package, led to the predictive linear discriminant analysis models. The predictive performance of each linear discriminant analysis model was evaluated by ROC curve analysis (Zweig et al. (1993) Clin. Chem. 39:561-577; Sing et al. (2005) Bioinformatics 21:3940-3941). The class prediction results from both the training and test data sets were grouped in modified 2×2 contingency tables and the statistical significance of the extent of agreement with clinical diagnosis was assessed by Fisher's exact test. Statistical test of correlation between DIGE and ELISA protein measurements was performed using R stats package.

Results

Plasma Protein Profile Distinguishes SJIA Flare from Quiescence

To determine the plasma protein profile associated with SJIA disease activity, we studied matched-pair plasma samples from 10 SJIA subjects at disease flare (F) and quiescence (Q). As expected, there were significant differences in laboratory values (leukocyte count, platelets, and sedimentation rate) and daily prednisone dose between the SJIA flare and quiescent samples (Table 1A).

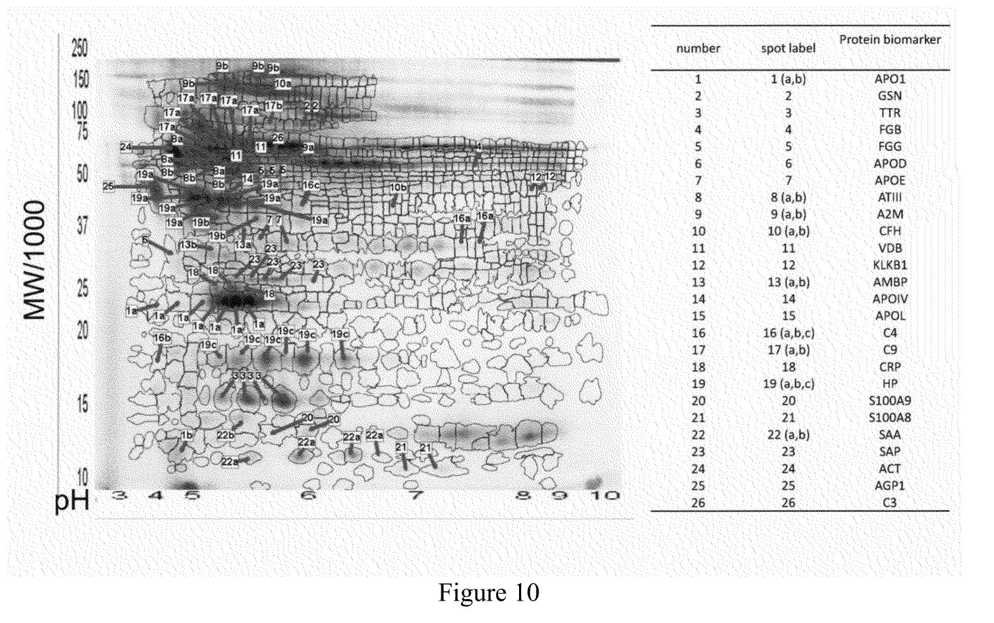

To prepare samples for 2D-DIGE, we depleted 6 abundant plasma proteins (see Materials and Methods), thereby enriching for lower abundance plasma proteins that are more likely to be biomarkers (Jacobs et al. (2005) J. Proteome Res. 4:1073-1085; Roche et al. (2009) J. Proteomics 72:945-951). We carried out 2D-DIGE on a mixture of equal amounts of protein from F and Q samples of each subject. Across all 10 gels, there were 889 plasma protein spots detected in the 2D-DIGE analyses. After normalization and manual review for candidate differentially expressed spots, we chose 96 protein features for identification by MALDI-TOF/TOF MS. Seven protein spots, representing hemoglobin and carbonic anhydrase from red blood cells, were removed from further analyses, leaving 89 identified protein features (FIG. 10).

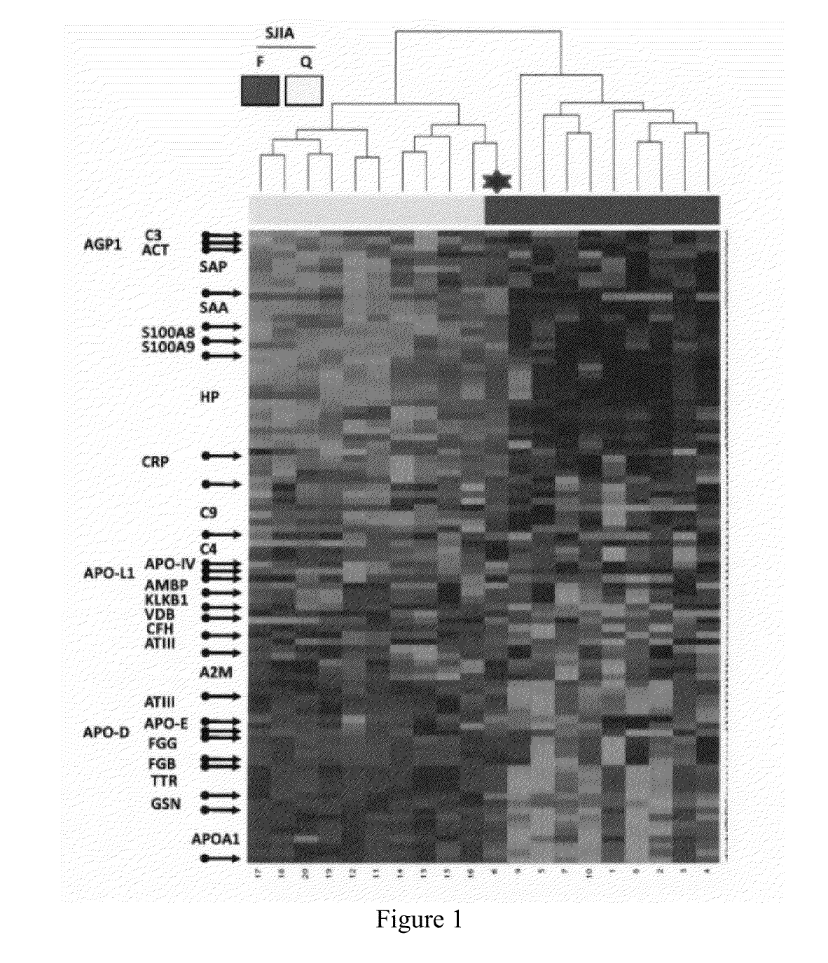

To assess the association of disease status with expression patterns of these 89 protein spots, we used normalized volume data from the 20 SJIA samples and performed unsupervised hierarchical clustering analysis with heatmap plotting (FIG. 1). The analysis shows 2 major clusters reflecting F and Q samples, indicating a flare “signature” in plasma. One F sample clustered with the Q branch. Clinical data suggested that the plasma protein expression pattern reflected reduced disease activity in advance of clinically detectable improvement (see discussion). The Q samples formed 2 subclusters. Clinical and demographic data (age, ethnicity, treatment response to MTX, TNF, or IL1RA, steroid dependence, poly/monocyclic course, joint damage) from these 2 subclusters did not identify obvious differences between them. However, the subgroups differed in the length of time (number of days) since SJIA was active, consistent with the possibility that particular plasma proteins (e.g., apoA1, some haptoglobin species) normalize faster than others as disease is becoming quiescent.

### Significant Differences in SJIA Flare Versus Quiescence Patterns

Mass spectrometric identification revealed that the selected 89 protein spots represented 26 plasma proteins: alpha-1-antichymotrypsin (ACT), alpha-1-acid glycoprotein (AGP1), alpha-2-macroglobulin (A2M), inter-alpha-trypsin inhibitor light chain (AMBP), apolipoprotein A1 (APO A-I), apolipoprotein A-IV (APO A-IV), apolipoprotein D (APO D), apolipoprotein E (APO E), apolipoprotein L1 (APO L1), antithrombin III (ATIII), complement C3 (C3), complement C4 (C4), complement C9 (C9), C-reactive protein (CRP), fibrinogen β (FGB), fibrinogen γ (FGG), gelsolin (GSN), complement factor H (CFH), haptoglobin (HP), kininogenin (KLKB1), calgranulin A (S100A8/MRP8), calgranulin B (S100A9/MRP14), serum amyloid A (SAA), serum amyloid P (SAP), transthyretin (TTR), vitamin D binding protein (VDB). We determined the extent of F versus Q differences in levels of expression (normalized volumes) of the 89 spots using Student's T-Tests. The results indicate that F versus Q differences are statistically significant (P value<0.05) for approximately ⅔ of the spots (59/89), representing 18 proteins and corroborating the generation of F and Q groups by cluster analysis of the 2DDIGE data.

### Optimization of the SJIA 2D-DIGE Flare Signature

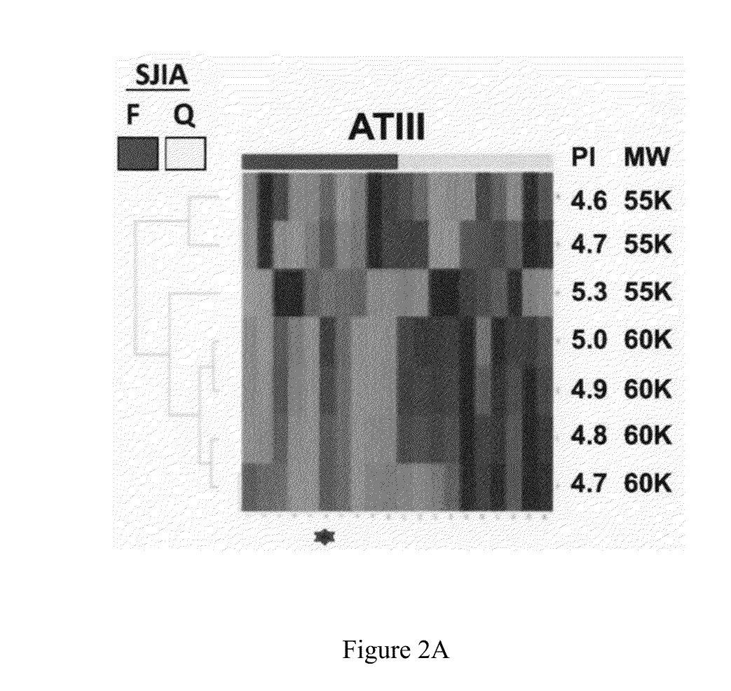

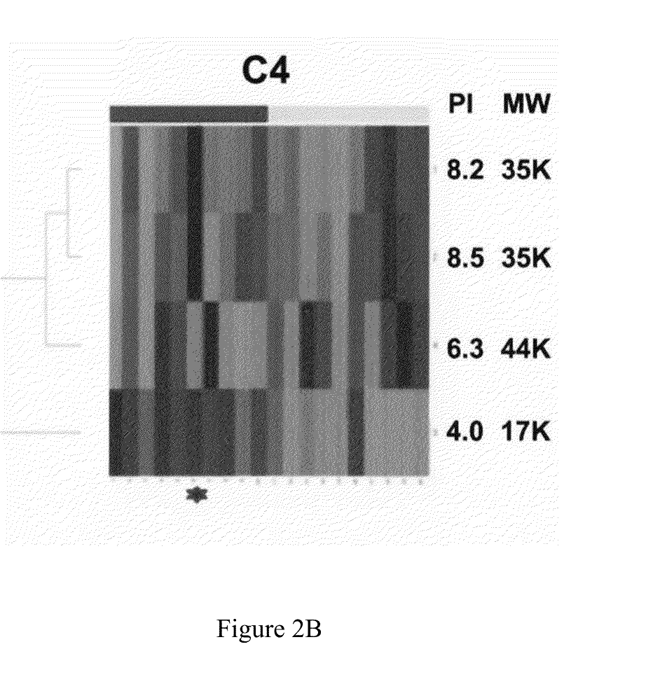

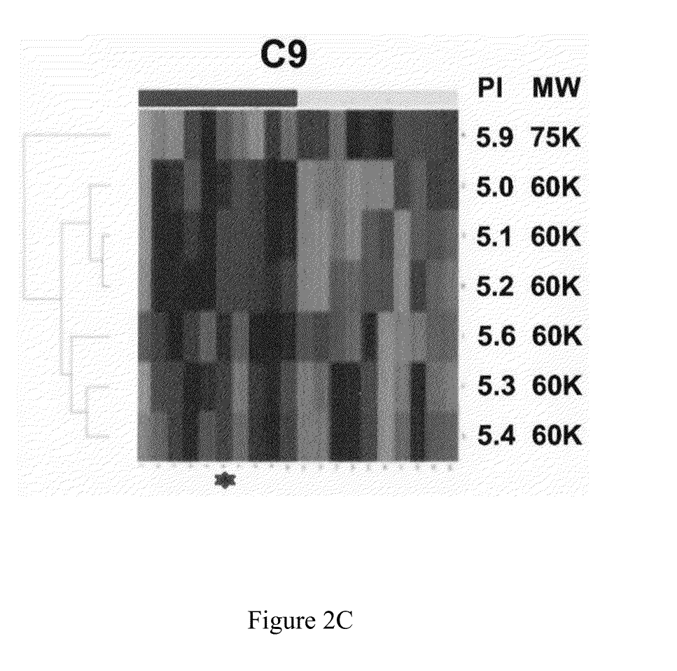

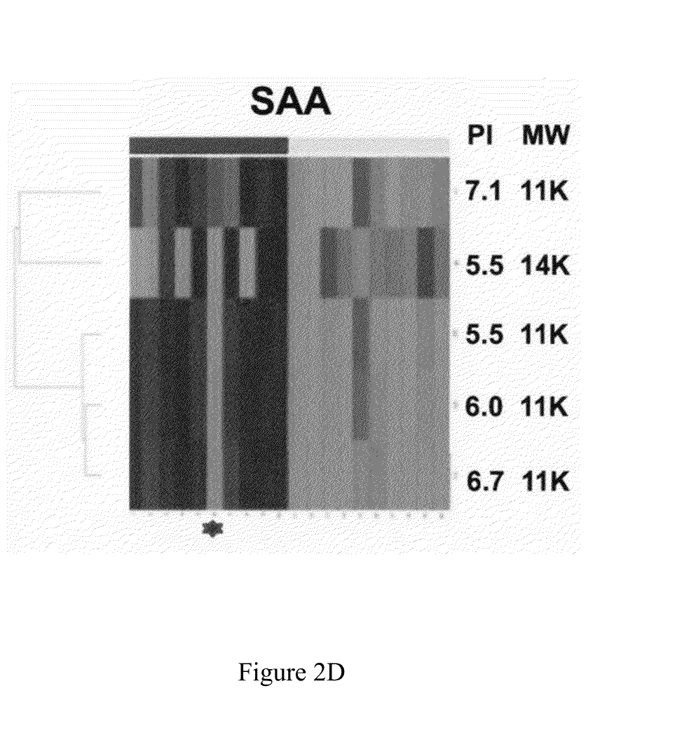

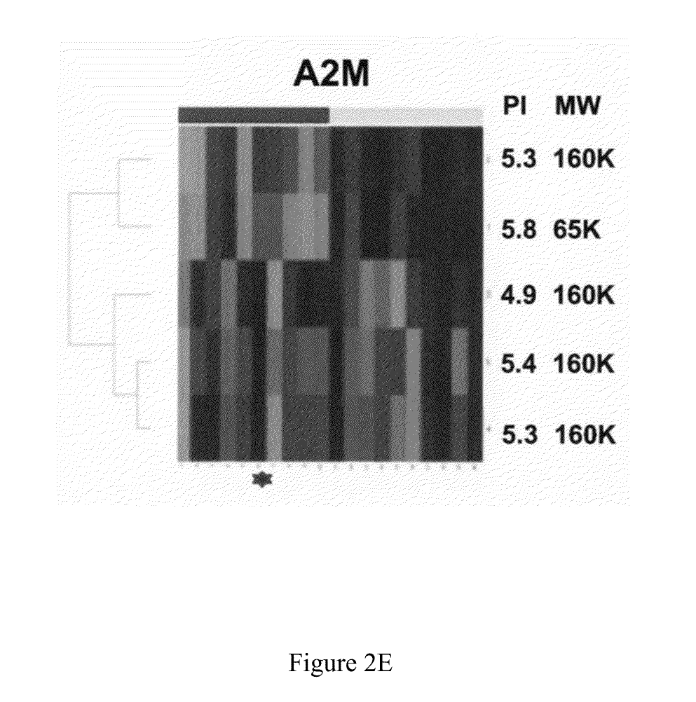

We noted that not all spots from the same protein had the same pattern of expression (FIG. 1), and Student's t test analyses revealed that different species of the same protein with different molecular weights (MW) and isoelectric points (pI) discriminated SJIA F and Q with different statistical significance. We hypothesized that the optimal SJIA flare signature would utilize a subset of protein features. For example, shown in FIG. 2 are heatmaps based on normalized volumes of spot sets from ATIII, C4, C9, SAA and A2M, with their estimated MW and pI values indicated. Three of four ATIII 60 kD spots (but not the most acidic species), give a more discriminating pattern for SJIA F versus Q than the three 55 kD spots. The discriminating spots are reduced at flare. The C4 spot at 17 kD, presumably a degradation product of C4, is the most discriminating and is generally increased in flare. The C9 75 kD spot generally is reduced at flare, whereas the C9 60 kD species are increased at flare, the most discriminating moieties being 60 kD spots with pIs of 5.1 and 5.2. For SAA, the more acidic 11 kD species, probably representing the mature protein, are the most discriminating, and are at higher abundance at SJIA F versus Q. The A2M 65 kD species, which is smaller than the mature protein, is a better discriminator of SJIA F and Q than the species at 160 kD. These findings support the notion that certain protein derivatives have strong discriminating power and that an optimized SJIA flare signature includes such features. Notably the flare sample misclassified as quiescent, indicated by the star in FIG. 2, expresses levels of the informative ATIII derivatives and of the informative SAA derivatives that correlate with quiescent status; generation of these protein species appears to be an early sign of reduced disease activity.

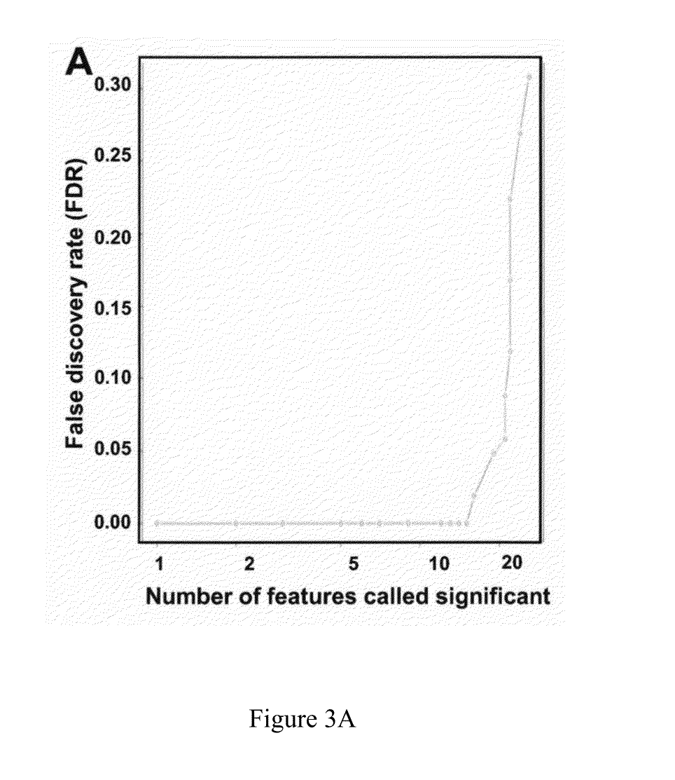

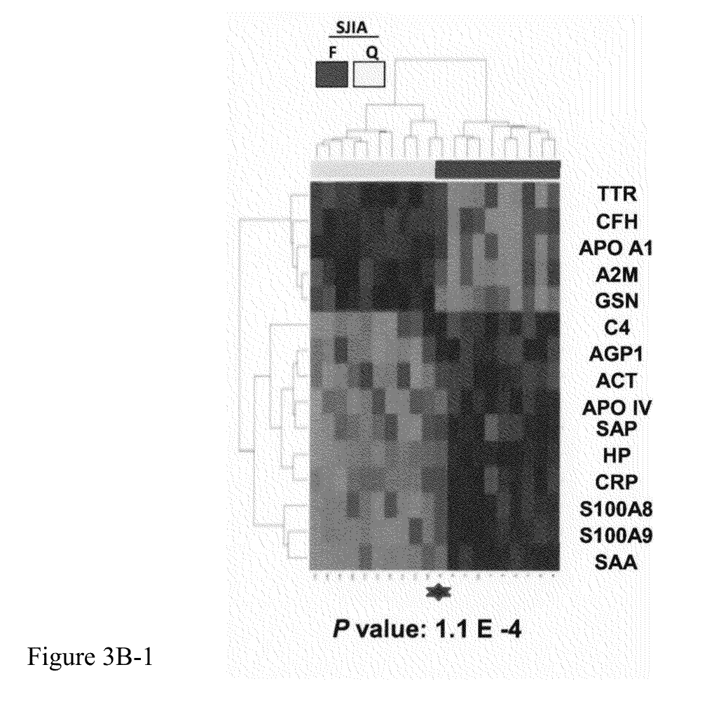

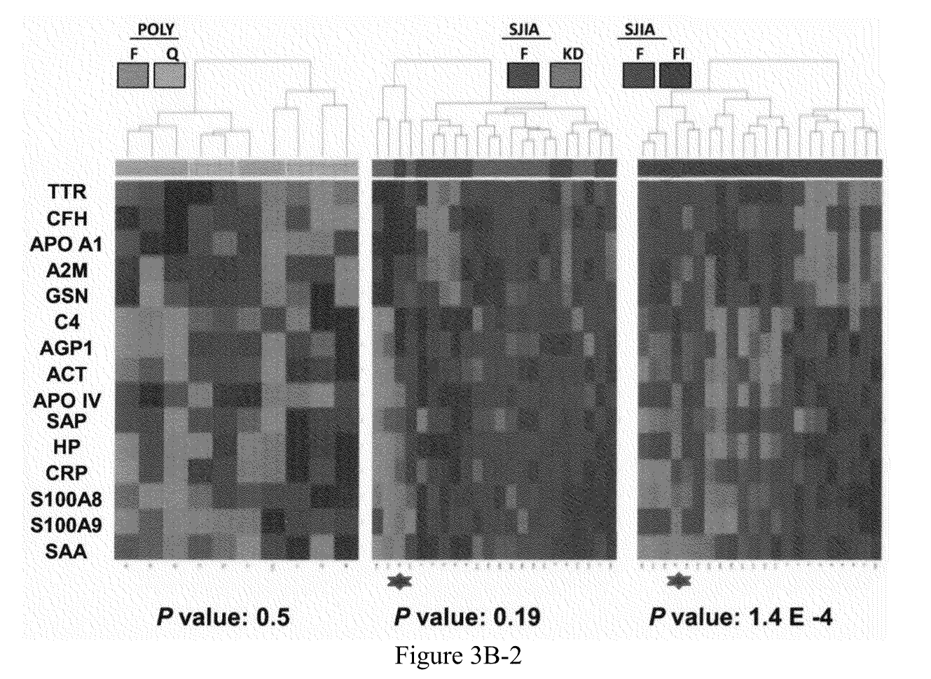

To select the panel of features with strongest discriminating power between SJIA F and Q, we applied the nearest shrunken centroid (NSC) algorithm (Tibshirani et al. Proceedings of the National Academy of Sciences of the United States of America 99:6567-6572) to normalized volumes of the most discriminating spots (lowest P value) for each of the 26 proteins from the 2D-DIGE analysis. False discovery rate (FDR) analysis showed significant FDR increase with feature sets larger than 15 (FIG. 3A, left). We used unsupervised clustering to analyze the top 15 protein spots (from TTR, CFH, APOA1, A2M, GSN, C4, AGP1, ACT, APOIV, SAP, HP, CRP, S100A8, S100A9 and SAA) as shown in the heatmaps in FIG. 3B. These proteins demonstrated the ability to distinguish SJIA F and Q robustly. To assess the specificity of this panel, we tested its ability to distinguish poly JIA F versus Q. In contrast to the effective discrimination between SJIA F and Q (P=1.1×10−4), the same protein spots discriminated poorly between poly JIA F and Q (P=0.5). The panel discriminated SJIA F from FI (P=1.4×10−4), but did not discriminate SJIA F from KD (P=0.19). Thus, the specific plasma features that distinguish active from inactive SJIA can also distinguish SJIA from the more localized inflammation of poly JIA and from the milder inflammation associated with acute febrile illness, but not from the more aggressive systemic inflammation of KD.

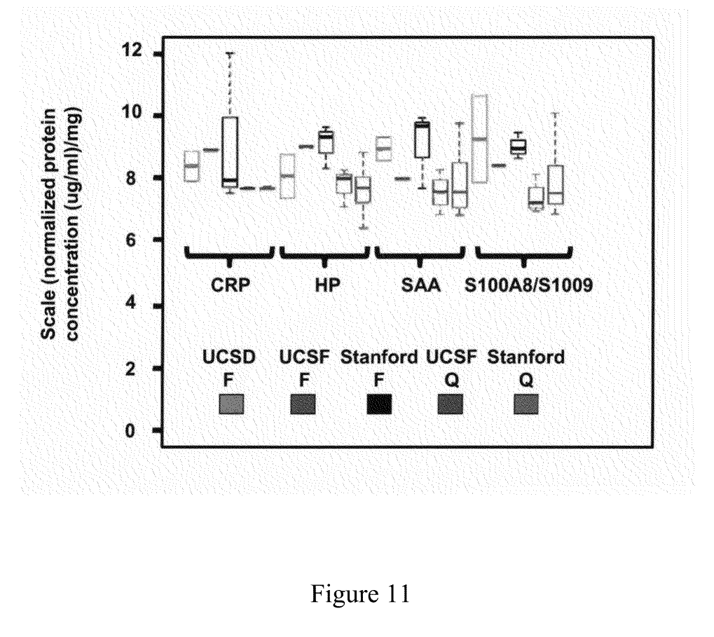

Center bias concerns were addressed, using ELISA assays for 4 of the signature proteins (CRP, HP, SAA and S100A8/S100A9) assays (FIG. 11). The results indicated that samples from the three clinical centers (Stanford University, UCSD and UCSF) have reproducible protein abundance patterns differentiating SJIA F and Q, which argues against the differences in SJIA and FI patterns being due to site of sample collection.

### ELISA-Based SJIA Flare Biomarker Panel

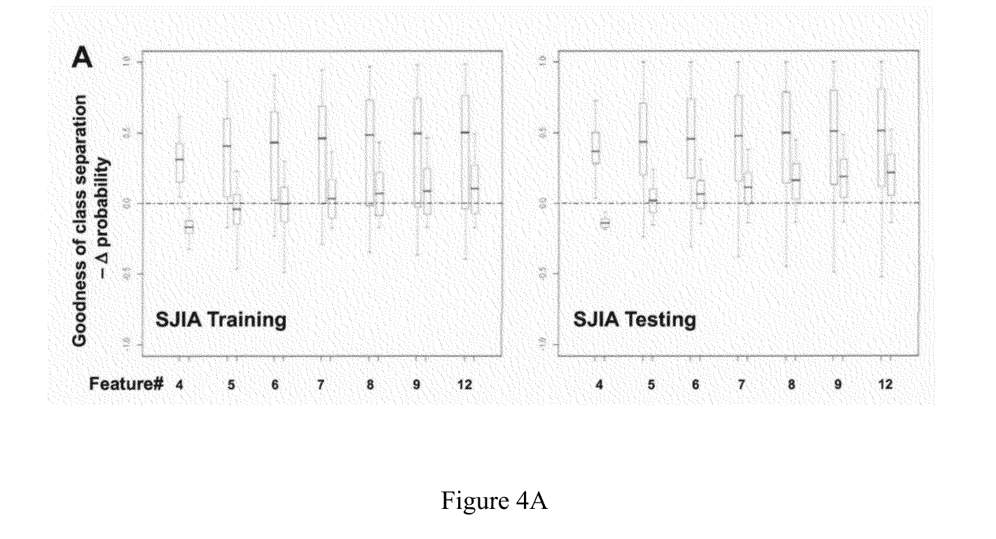

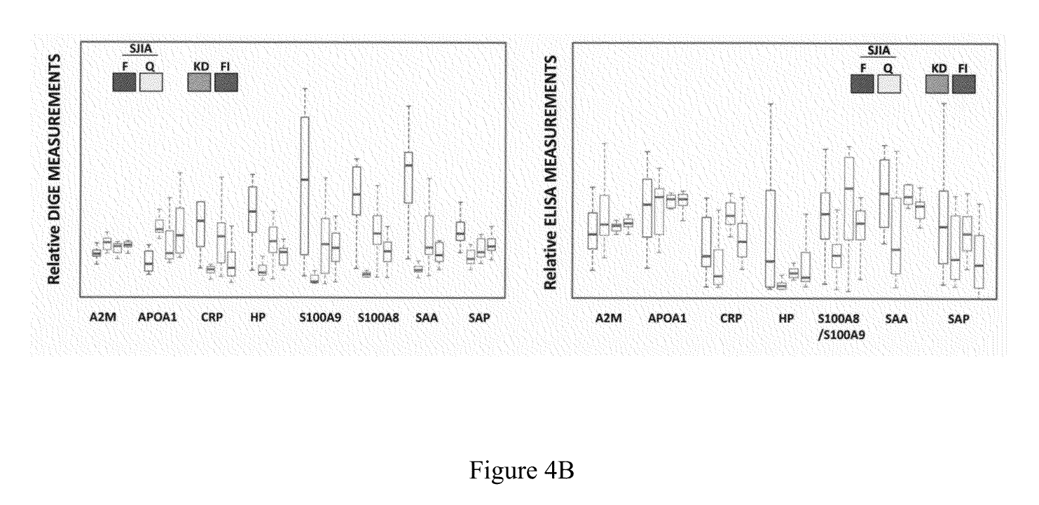

We were interested in whether the SJIA F panel could lead us to an immediate practical clinical tool, based on available antibodies and ELISA assays. We selected a panel of 9 of the 15 SJIA F (vs. Q) proteins (SAP, SAA, S100A8, S100A9, HP, CRP, A2M, APO-A1, TTR) and the S100A8/S100A9 complex. We also included ATIII, which showed discriminating power in the 2D-DIGE and S100A12, a protein of the S100 family found by other investigators to increase at SJIA flare (Wittkowski et al. (2007) Arthritis Rheum. 56:4174-4181; we confirmed the S100A12 association with SJIA F in our cohort by ELISA, data not shown). We performed ELISA assays on a training set of samples, 12F/12Q (10/24 samples are matched from 5 subjects), and a test set, 10F/10Q (8/20 samples are matched from 4 subjects). Using data from these assays, we built classifiers with various subsets of the 12 ELISA assays. We sought to identify a biomarker panel of optimal feature number, balancing the need for small panel size, accuracy of classification, goodness of class separation (F vs. Q), and sufficient sensitivity and specificity. Goodness of separation is defined by computing the difference (A) between discriminative scores, calculated as estimated probabilities (Tibshirani et al. (2002) Proc. Natl. Acad. Sci. U.S.A. 99:6567-6572). When class is predicted correctly, A probability is the difference of the highest and next highest probability; when predicted incorrectly, A probability is the difference of the probability of the true class and the highest probability, which will be negative. Shown in FIG. 4A are the SJIA F and Q box-whisker graphs. Boxes contain the 50% of values falling between the 25th and 75th percentiles; the horizontal line within the box represents the median value and the “whisker” lines extend to the highest and lowest values. The analysis revealed 7 to be the smallest panel size for which the “box” values of goodness of separation are positive for both SJIA F and Q, in both training and testing data sets.

The 7-ELISA panel consists of A2M, APO-AI, CRP, HP, S100A8/S100A9, SAA, and SAP. We compared the results obtained by 2D-DIGE and ELISA assays (FIG. 4B). The boxwhisker graphs illustrate the distribution of values for each of the panel proteins. The trends for relative abundance of each biomarker across SJIA F, SJIA Q, KD and FI clinical classes are consistent between DIGE and ELISA assays. We tested for correlation between the DIGE and ELISA measurements by Kendall's tau, which is a rank-based statistic. This revealed P=0.02, indicating that ELISA and DIGE observations are statistically correlated, and therefore ELISA assays validate the DIGE observations.

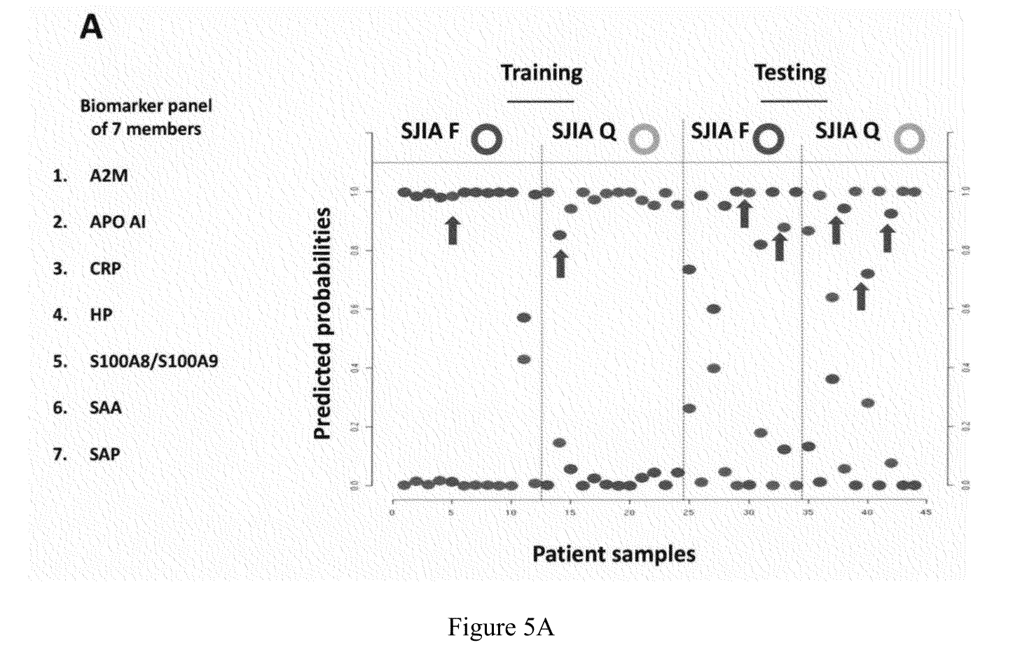

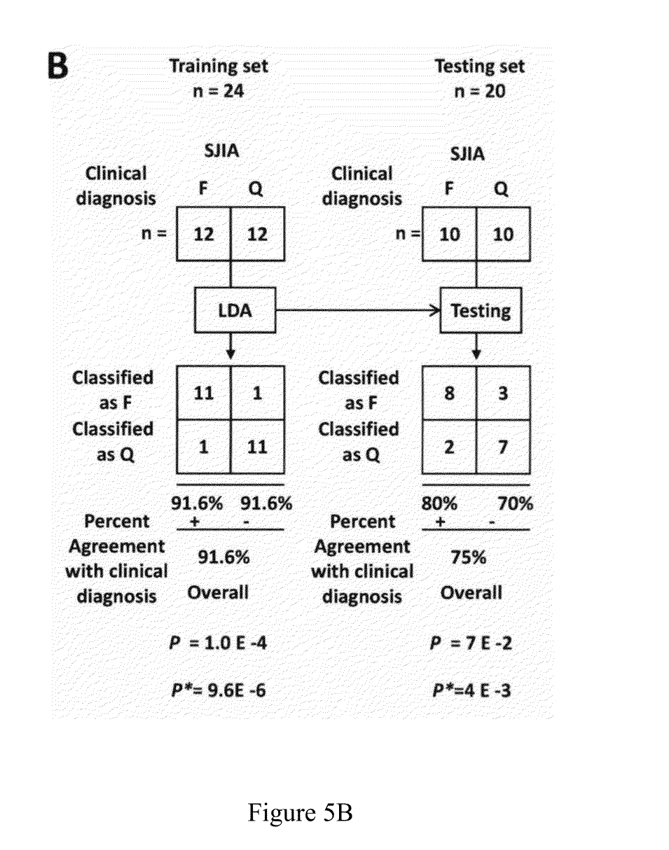

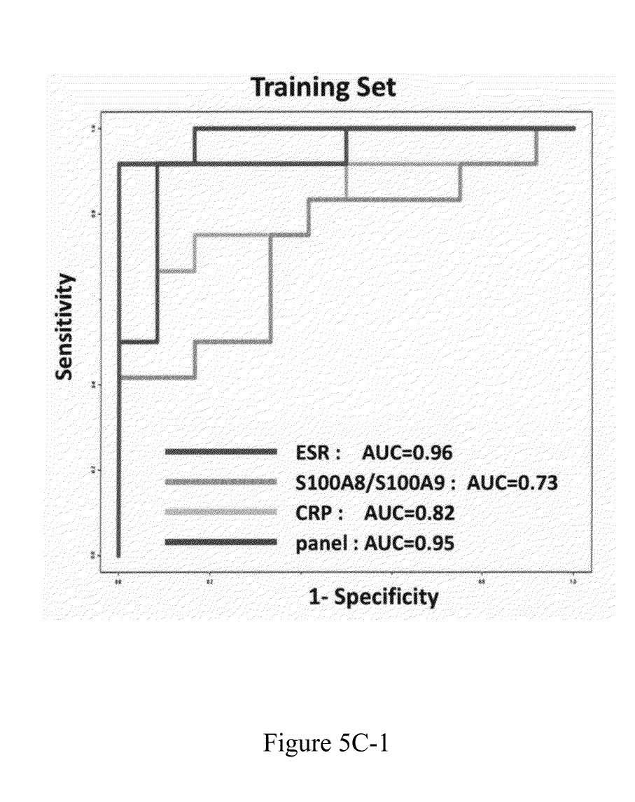

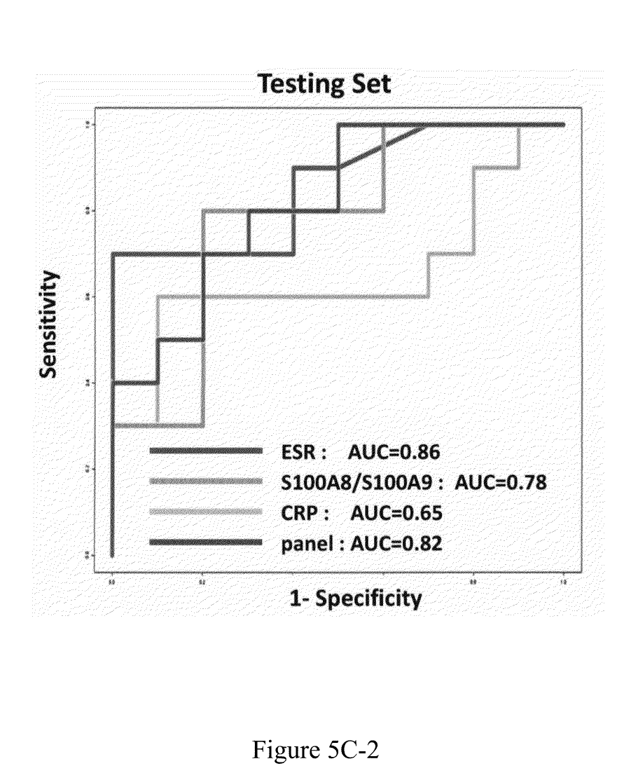

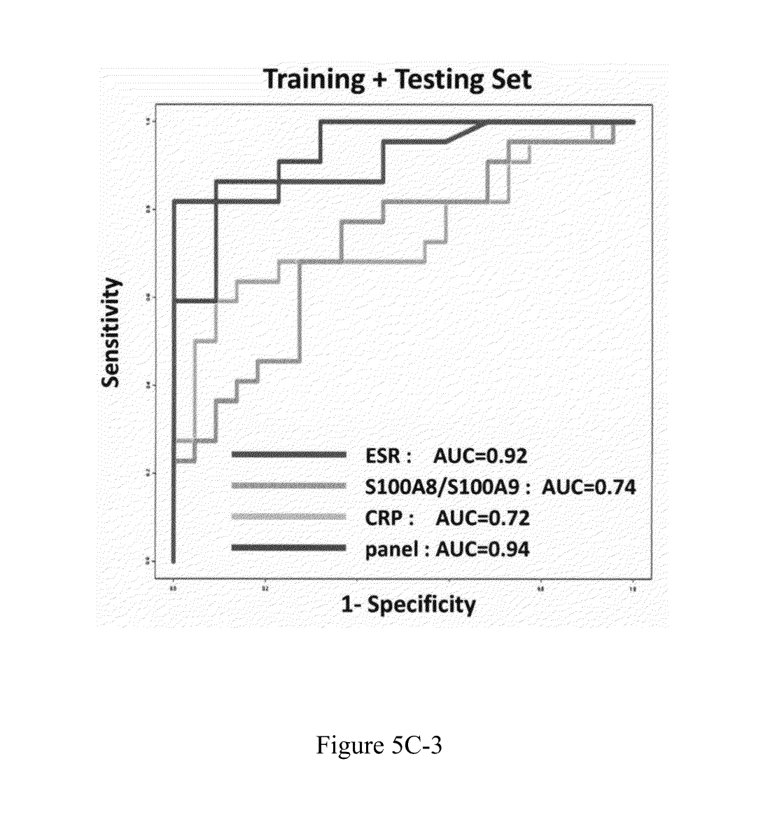

To gauge the efficacy of the 7 ELISA panel as a classifier for SJIA disease activity, we performed linear discriminant analysis (FIG. 5). This yielded 22/24 assignments that agree with clinical assessment in the training sample set and 15/20 assignments that agree with clinical assessment in the test sample set (FIG. 5A). The probabilities associated with these classification choices are plotted. The maximum estimated probability for each of the wrongly classified samples is marked with an arrow. As shown in FIG. 5B, the 7 ELISA panel-based algorithm classified the training F samples with 91.6% agreement and the Q samples with 91.6% agreement with clinical class, with P=1.0×10⁻⁴. With the test set data, the F samples were classified with 80% agreement and Q samples with 70% agreement with the clinical diagnosis, with P=0.07. The misclassified patient in the SJIA Q training group was noted to flare clinically four weeks after her quiescent sample was drawn, and one of the misclassified subjects in the SJIA F test group was noted to be in clinical quiescence by his next visit, 2.5 months later. These findings again suggested that the classifier detects changes of disease state in advance of clinically detectable changes. Another misclassified subject in the SJIA F test group was noted to have concomitant (probable viral) gastroenteritis, raising the possibility that the classifier may distinguish fever and rash due to disease flare from that due to a viral or infectious process. Recalculation of P values for accuracy of classification, based on removing these 3 subjects, results in statistically significant values for both data sets (FIG. 5B, P*=9.6×10⁻⁶; P*=4×10⁻³). A fourth misclassified SJIA F sample was from a subject with active arthritis without systemic systems, suggesting that the panel is weighted toward detection of activity of the systemic manifestations of SJIA.

For both training and test data sets, ROC analyses (Zweig et al. (1993) Clin. Chem. 39:561-577; Sing et al. (2005) Bioinformatics 21:3940-3941) were performed to assess the performance of the SJIA flare classification algorithm and compared to ESR, CRP, or S100A8/S100A9 (FIG. 5C). The ROC analyses yielded AUCs of 0.95 for our panel, ESR 0.96, S100A8/S100A9 0.73, CRP 0.82 with the training data set, and with the test data set, our panel 0.82, ESR 0.86, S100A8/S100A9 0.78, CRP 0.65. The final classifier using observations from the combined training and test sets yielded AUCs for our panel of 0.94, ESR 0.92, S100A8/S100A9 0.74 and CRP 0.72, respectively. These analyses indicated that our panel was comparable to ESR for detection of flare, but better than either CRP or S100A8/S100A9 in SJIA F/Q discriminations.

Distinguishing SJIA Flare from Poly JIA Flare or Acute KD or FI Using ELISA Panel

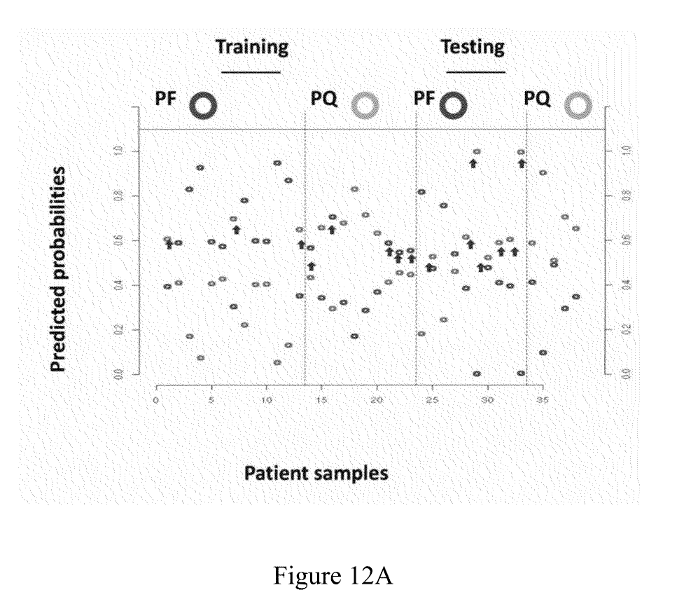

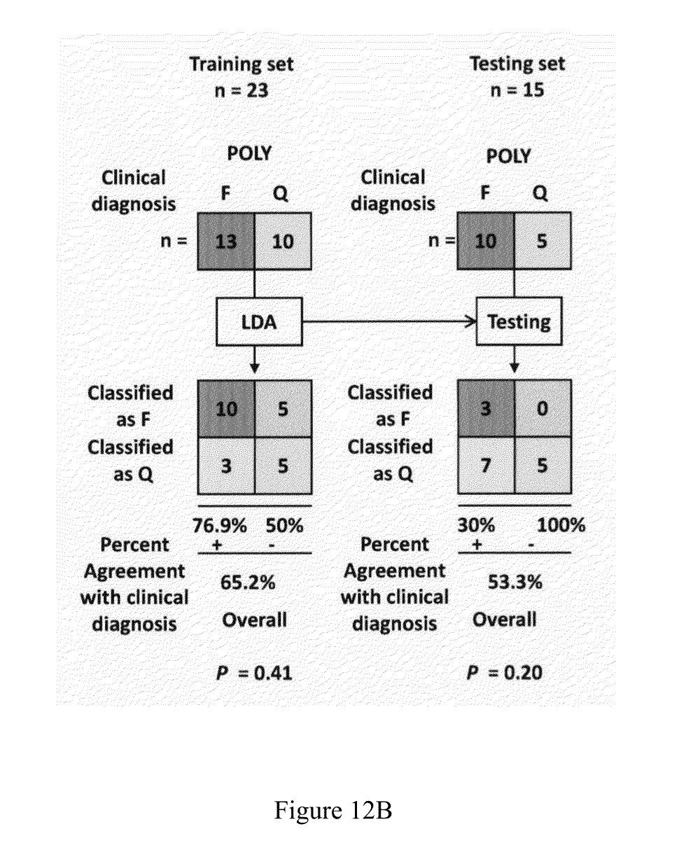

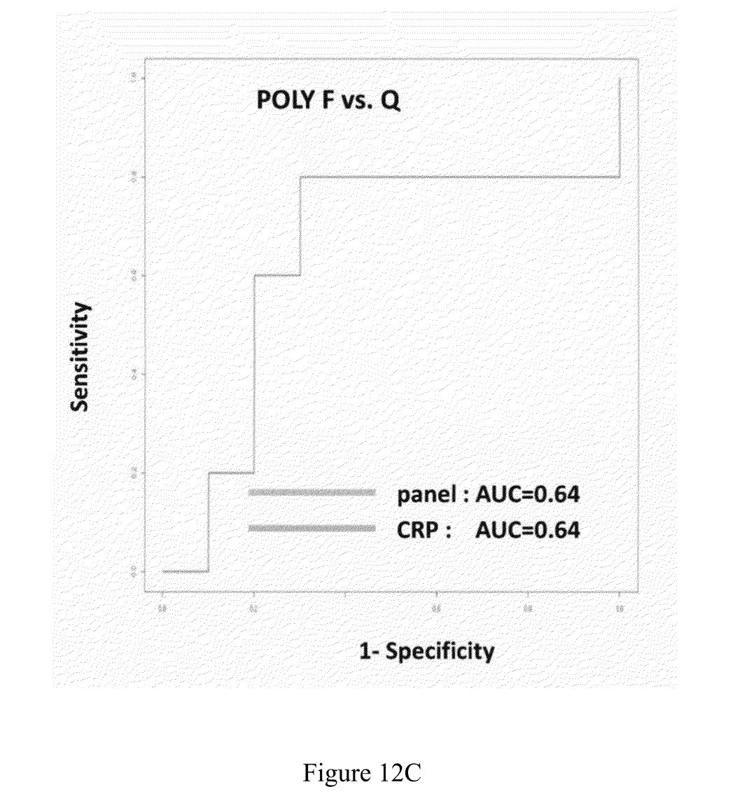

To test the 7-biomarker SJIA flare panel in poly JIA F vs. Q discrimination, we performed ELISA assays on a training set of poly JIA samples, 13F/10Q (10/23 samples are matched from subjects), and a test set, 10F/5Q (4/15 samples are matched from 2 subjects). The linear discriminant classification results are plotted (FIG. 12A) and shown in modified 2×2 contingency tables. Fisher's exact testing of the percentage of classifications that agree with clinical assessment indicated no statistical significance (P=0.41 for training data; P=0.2 for test data); ROC analyses showed AUC values of only 0.64 for both training and test sets. Thus, this panel is not a disease activity classifier for poly JIA in our cohort.

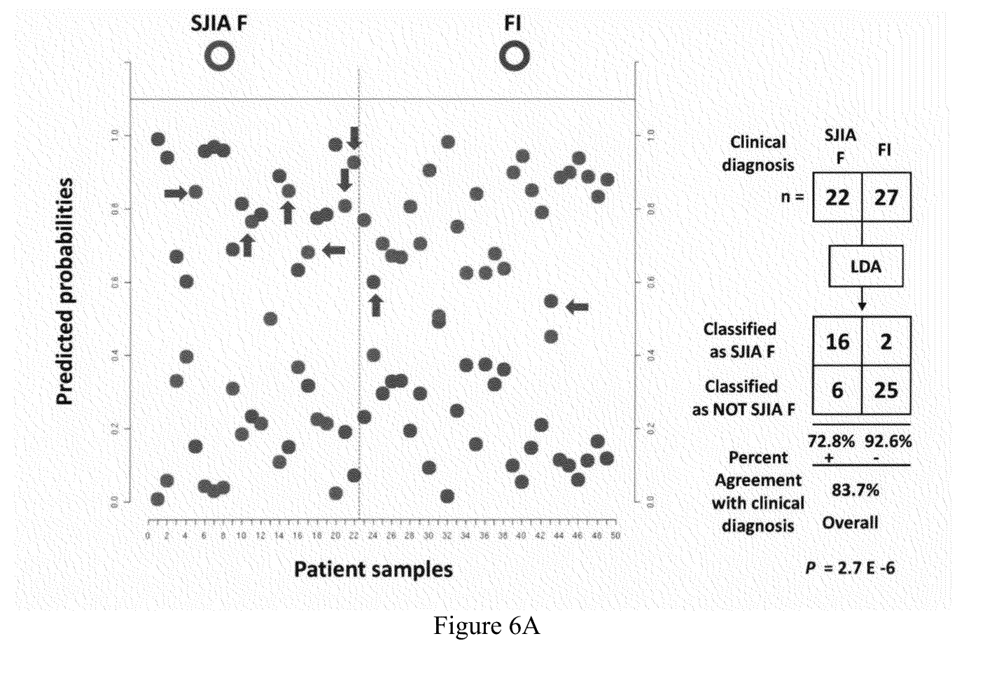

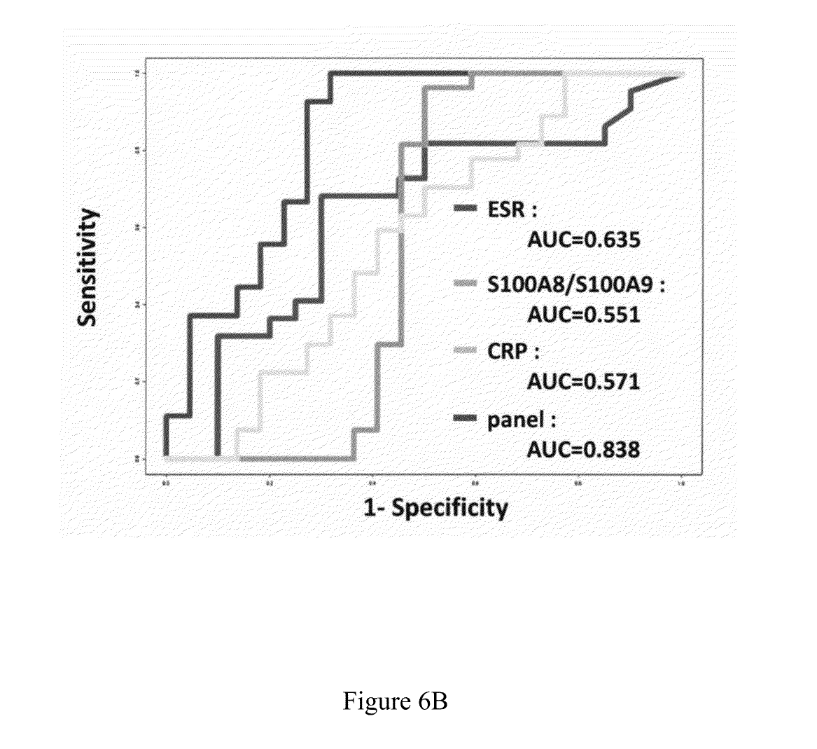

DIGE data indicated efficient discrimination between SJIA flare and the inflammation of acute FI (FIG. 3B). We asked whether the ELISA panel would be sufficient to identify these clinical conditions. The levels of the biomarkers in 49 samples (including 22 SJIA F studied in FIGS. 5 and 27 new FI subjects) were measured by the 7 ELISA assays. FIG. 6A plots the discriminant probabilities of the ELISA-based classifier for the assayed subjects. 16/22 SJIA F subjects were classified correctly as SJIA, and 25/27 FI samples were classified as FI. Fisher exact test of the 2×2 contingency tables of classification results yielded P=2.7×10⁻⁶, indicating the effectiveness of biomarker-based classifier in discriminating SJIA F from FI. Similar results were obtained using 22 different SJIA F samples taken from the same subjects at different visits (not shown). Among the misclassified SJIA samples in FIG. 6A are 3 SJIA F samples that are misclassified in FIG. 5A and are discussed above. The efficacy of the ELISA panel as a classifier of SJIA versus acute febrile illness was further confirmed by comparative ROC analyses, which gave AUC values for the SJIA flare panel (0.838) that were higher than AUC values for S100A8/S100A9 (0.551), ESR (0.635) and CRP (0.571) alone (FIG. 6B). These results support the potential clinical utility of the SJIA flare panel for separating SJIA F from the inflammation of acute FI.

Detection of Impending Flare with the SJIA Flare ELISA Panel

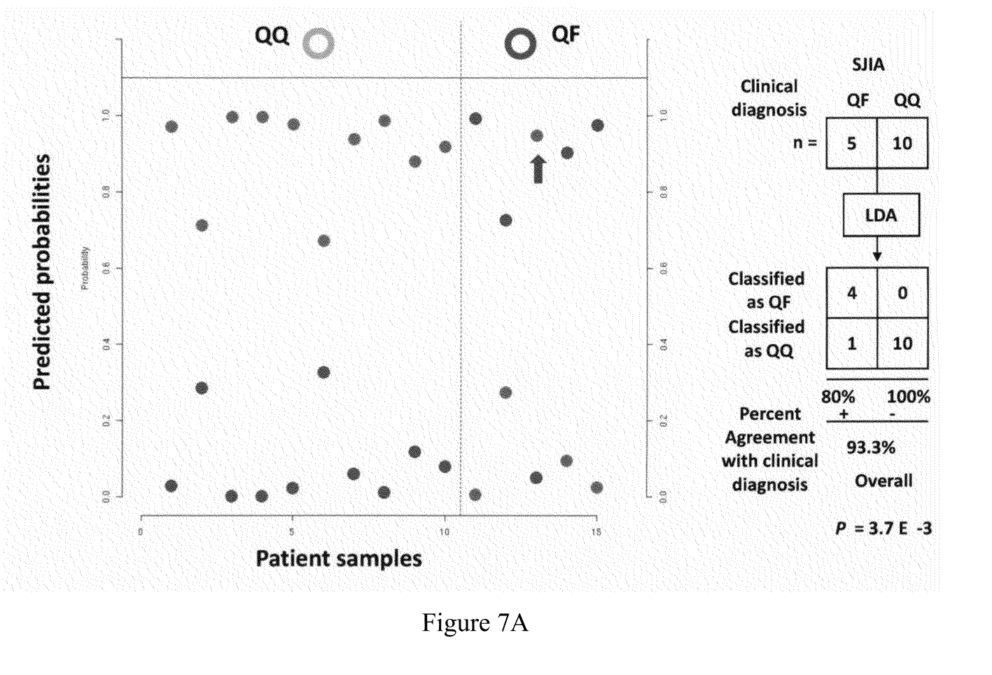

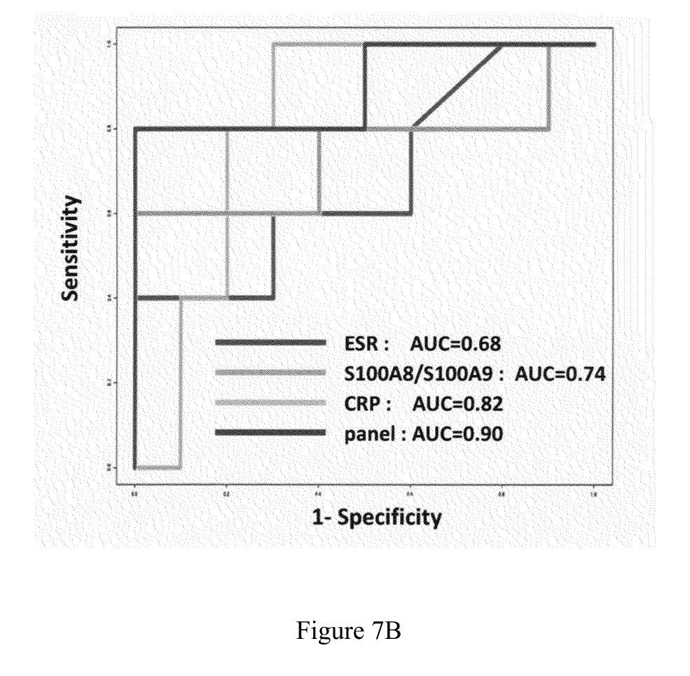

To test whether the ELISA-based biomarker panel has the capacity to detect “early” SJIA flare prior to clinically detectable disease activity, two sets of SJIA samples were compared: 5 SJIA quiescent samples (QF) drawn within 2-9 weeks prior to a clinical flare and samples from 10 SJIA quiescent controls (QQ), whose disease was quiet for 6 months before and after sample collection. All samples in this QQ and QF stratification analysis were different from those used in the previous analyses. ELISA data sets were used to develop a binary classifier (QQ versus QF). FIG. 7A plots the classification results and shows that both QQ and QF samples have clear separation between the highest and next highest probability for the classifier assignment. Only one QF sample was misclassified and is marked with an arrow. A Fisher exact test of the 2×2 contingency tables of classification results yielded P=3.7×10⁻³, indicating the effectiveness of the classifier in prediction of impending SJIA flare. This was further confirmed by comparative ROC analyses, which gave AUC values of 0.90 for the SJIA flare panel, whereas other values were: ESR 0.68, S100A8/S100A9 0.74, CRP 0.82 (FIG. 7B). These results support the potential clinical utility of the SJIA flare panel in predicting impending SJIA flare.

### DISCUSSION

Our initial 2D-DIGE results indicated differential plasma protein profiles between active and inactive SJIA. To our knowledge, this is the first study to describe a unique proteomic profile of SJIA F using 2D-DIGE. Because greater than 50% of plasma protein content is accounted for by albumin and other abundant proteins, such as IgG and transferrin, we performed an initial depletion step, removing six of the most abundant proteins. This step allowed us to detect less abundant proteins, such as serum amyloid P (Huang et al. (2005) Electrophoresis 26:2843-2849). The DIGE technique has a dynamic range of about 5 orders of magnitude in protein concentration (Gibson et al. (2009) J. Proteomics 72:656-676), whereas plasma protein concentrations vary over about 10 orders of magnitude, with the highest concentrations reaching mg/ml (Anderson (2002) Mol. Cell. Proteomics 1:845-867). Even with the depletion step, protein detection by our 2D-DIGE system is limited to proteins whose plasma concentrations are greater than 10 μg/ml, clearly influencing the composition of the SJIA flare signature we detected. In addition, potentially informative low molecular weight proteins may bind to albumin and thus be removed at the depletion step (Tirumalai et al. (2003) Mol. Cell. Proteomics 2:1096-1103). Nonetheless, as levels of some abundant plasma proteins are reduced (e.g., APO A-1, TTR) or increased (e.g. CRP) significantly during inflammatory states, and other proteins that are not found in normal control plasma rise to a level detectable by 2D-DIGE, such as S100A9, a flare signature is observable. Moreover, specific protein species are altered in abundance during active SJIA and are sufficient to produce signatures that robustly differ from the protein pattern at SJIA quiescence and from other inflammatory conditions we tested (see more below).

From the 2-D DIGE, we evaluated 89 spots, representing 26 proteins, as candidate components of the SJIA F flare signature. Among these, some proteins have individually been associated with SJIA flare, such as SAA, CRP and the inflammation-associated S100A8/S100A9 complex (Frosch et al. (2009) Arthritis Rheum. 60:883-891; De Beer et al. (1982) Lancet 2:231-234; Wu et al. (2007) Clinical and Experimental Rheumatology 25:782-785). In addition, our observation of reduced levels of APO A-1 at SJIA flare confirms previous investigations of JIA subjects (Tselepis et al. (1999) Arthritis Rheum. 42:373-383). The fact that these (expected) proteins were identified by our analyses increases confidence in the use of DIGE as a platform to detect plasma proteins differentially expressed in association with SJIA disease activity.

Particular isoforms/derivatives of 15 proteins gave rise to a robust SJIA flare signature that differentiated flare from quiescence and from acute febrile illnesses. These 15 proteins can be assigned to different functional groups, including proteins involved in the classical acute phase response, the innate immune system (S100 proteins), the complement cascade, the coagulation system and lipid/cholesterol metabolism. There is the substantial evidence supporting crosstalk between these pathways in inflammatory states. For example, CRP, a quintessential positive acute phase protein, binds molecular patterns typically found on the surface of pathogens and also activates the classical complement pathway (Black et al. (2004) J. Biol. Chem. 279:48487-48490; Dayer (2007) Nature Reviews Rheumatology 3:512-520). A2M, a thrombin (and other protease) inhibitor, is also a component of the innate immune system, acting as a scavenger of novel proteases introduced by pathogens (Armstrong et al. (1999) Dev. Comp. Immunol. 23:375-390). APO A-1, the major protein component of high density lipoprotein (HDL), also has anti-inflammatory and anti-thrombotic properties (Dayer et al. (2007) Nature Reviews Rheumatology 3:512-520; Yui et al. (1988) Journal of Clinical Investigation 82:803-807). Acute phase HDL, where SAA is exchanged for APO A-1, are lipid transport particles, but also function in innate immune responses, for example, by promoting monocyte chemotaxis (Navab (2005) Trends in Cardiovascular Medicine 15:158-161). APO A-IV, the major protein component of intestinal triacylglycerol-rich lipoproteins, is a positive acute phase protein involved in lipid homeostasis, but also reduces Toll-like receptor 4-induced pro-inflammatory cytokines (Khovidhunkit (2004) Atherosclerosis 176:37-44; Recalde et al. (2004) Arterioscler. Thromb. Vasc. Biol. 24:756-761).

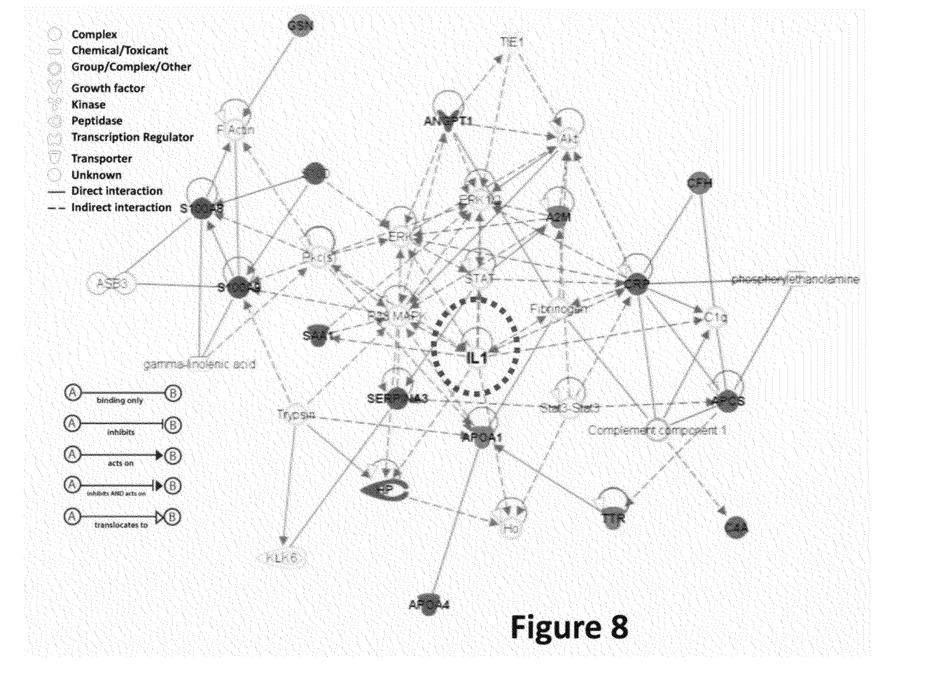

We analyzed the 15 proteins that are significantly differentially expressed in SJIA flare as a composite, using Ingenuity Pathway Analysis software (IPA version 7.6, Ingenuity Systems, Inc., Redwood City, Calif.). Strikingly, as shown in FIG. 8, all 15 proteins are linked in one network by the software, with the central molecular driver identified as IL-1. Acute phase response signaling is identified as the top canonical pathway with a P value of 1.38×10⁻¹⁴. IL-1β and TNFα, pro-inflammatory cytokine products of monocyte/macrophages, are known to stimulate IL-6 production by monocyte/macrophages and endothelial cells. These cytokines, and IL-6 especially, act on hepatocytes to induce production of classical acute phase proteins, such as SAA and CRP, complement components and fibrinogen and suppress production of proteins such as APO A-1 (Dayer et al. (2007) Nature Reviews Rheumatology 3:512-520). Notably, the evidence of IL-1 activity, as reflected in the pattern of proteins in SJIA plasma at flare, is consistent with recent reports of the therapeutic effects of IL-1 inhibition in SJIA patients (Pascual et al. (2005) Journal of Experimental Medicine 201:1479-1486; Yokota et al. (2008) Lancet 371:998-1006).

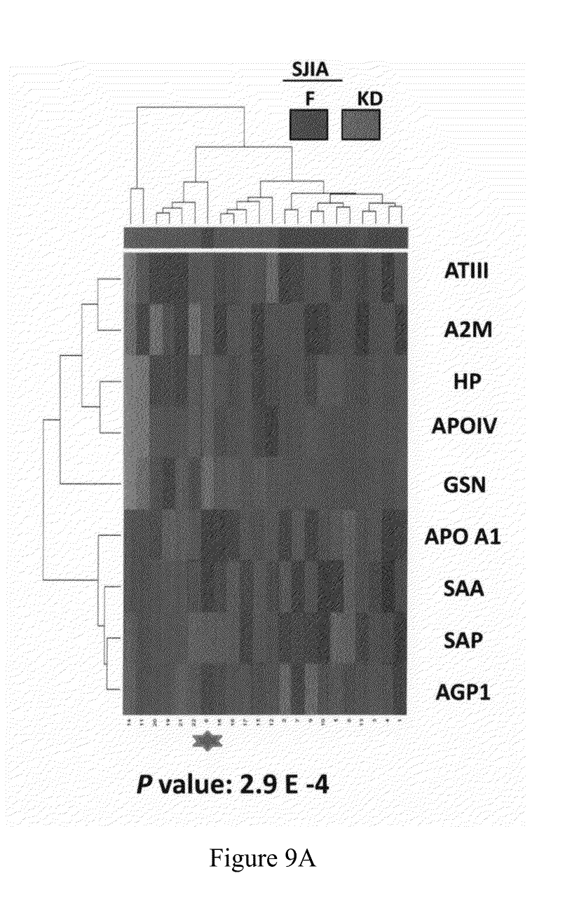

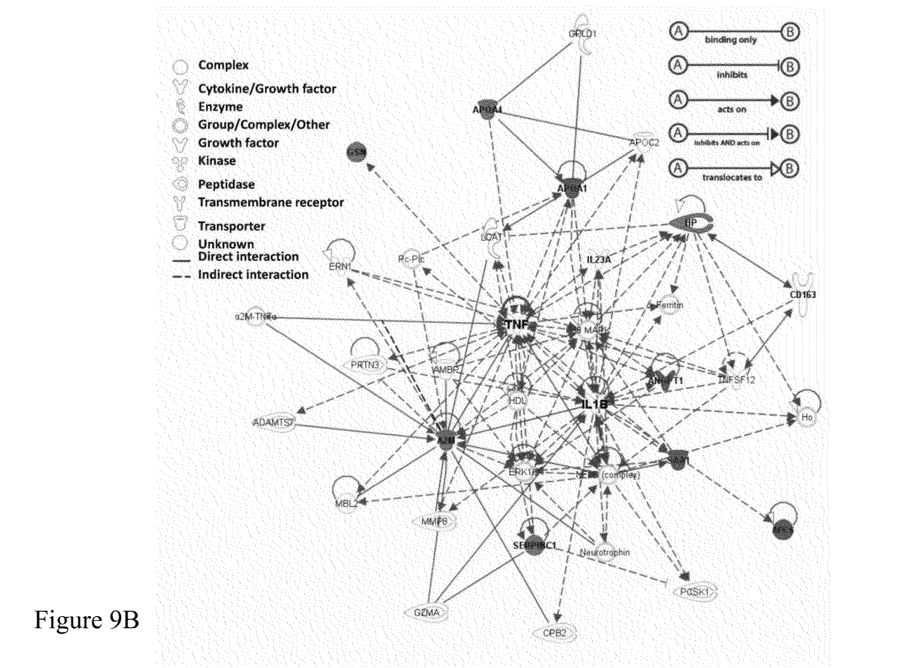

Our data show that the differentially expressed plasma proteins at SJIA F compared to Q have a substantial degree of specificity for SJIA F, compared to poly JIA F or FI; this is the case for both proteins detected by 2D-DIGE and by the ELISA panel. These observations corroborate other evidence indicating that specific patterns of acute phase reactants are associated with certain diseases (Braunwald (2008) N. Engl. J. Med. 358:2148-2159; Kawachi-Takahashi et al. (1975) International Archives of Allergy and Applied Immunology 48:161-170; Kawachi-Takahashi et al. (1974) Japanese Journal of Experimental Medicine 44:845-847; Bene et al. (2003) Digestive Diseases and Sciences 48:1186-1192). In a relevant example, Yu et al (Pediatric Allergy and Immunology (2009) 20:699-707) described a unique 2D protein fingerprint in KD versus non-KD febrile control subjects, with increases in protein spots, representing fibrinogen β and γ chains, α-1-antitrypsin, CD5 antigen-like precursor, and clusterin, and decreases in spots from immunoglobulin light chains. This pattern differs from SJIA flare, although we confirmed a significant difference in fibrinogen β between KD and FI, and noted a KD-specific increase in APO-D compared to FI subjects. Similarly, a study of gene expression in peripheral blood mononuclear cells (PBMC) showed that the list of genes differentially expressed in SJIA patients compared to controls had more overlap (35/286) with PBMC gene expression in an autoinflammatory condition (neonatal onset multisystem inflammatory disease, NOMID) than with PBMC gene expression in poly JIA (6/286) or KD (17/286) (Ogilvie et al. (2007) Arthritis and Rheumatism 56:1954-1965). Disease-associated variation in acute phase proteins implies their independent regulation and is thought to reflect differences in the driving cytokines and their endogenous modulators (Gabay et al. (1999) N. Engl. J. Med. 340:448-454). This idea finds support within childhood rheumatic diseases in the apparent roles for IL-1β and IL-6 in SJIA, as compared to TNFα/sTNFR in poly JIA or interferon α in SLE (Pascual et al. (2005) Journal of Experimental Medicine 201:1479-1486; Yokota et al. (2008) Lancet 371:998-1006; Prince et al. (2009) Ann. Rheum. Dis. 68:635-641; Pascual et al. (2006) Current Opinion in Immunology 18:676-682). Differences in profiles of PBMC transcripts or plasma proteins from active SJIA and acute KD are of particular interest because, at disease onset, these two conditions can present a diagnostic dilemma. To explore the possibility that a panel could be identified to directly distinguish SJIA F from KD, the gel spots discriminating between SJIA F and KD with Student's t test P value<0.05, were chosen for unsupervised analysis. A new panel of features from nine proteins (ATIII, A2M, HP, APOIV, GSN, APO A1, SAA, SAP, and AGP1) suggests that plasma profiles can identify 2 subsets of KD patients, one more similar to SJIA than the other (FIG. 9). This classifier uses different protein derivatives than the SJIA F vs. Q panel, although 8 source proteins are shared. When the changes in these source proteins are analyzed by Ingenuity, acute phase response signaling again is identified as the top canonical pathway function with P value=2.34×10⁻⁸. Interestingly, two new molecular links appear, suggesting processes that differ between SJIA and at least a subset of KD subjects: IL-23, a cytokine associated with Th17 cells, and CD163, a scavenger receptor on alternatively activated macrophages, CD163 is known to bind and clear haptoglobin/hemoglobin complexes and monocyte/macrophages expressing this receptor have been implicated in SJIA, particularly in association with a life threatening complication of the disease termed “macrophage activation syndrome” (Fall et al. (2007) Arthritis Rheum. 56:3793-3804).

We detected a highly discriminatory SJIA flare signature by identifying the particular protein species (spot) most highly associated with disease activity. Different post-translational modifications, particularly altered glycosylation, and/or proteolysis of plasma proteins associated with active disease most likely underlie this observation (Gabay et al. (1999) N. Engl. J. Med. 340:448-454; Wu et al. (2006) Journal of Proteome Research 5:651-658). These changes are likely cytokine-driven. For example, it is known that matrix metallo-proteinases (MMPs), especially MMP-1, -3, -9 and -13 are induced by IL-1β (Ge et al. (2009) Arthritis and Rheumatism 60:2714-2722; Lin et al. (2009) Cellular Signalling 21:1652-1662). In a separate study of low concentration plasma proteins, we have found that increased circulating MMP9 is associated with SJIA flare (Ling, X B et al, manuscript in preparation). More work is warranted to investigate the molecular events that generate specific protein modifications and intermediates in inflammatory states. Of note, increased levels of SAA-related derivatives are found in supernatants of IL-1β-activated human monocytes and are thought to reflect a block in SAA degradation (Migita et al. (2001) Clinical and Experimental Immunology 123:408-411). These in vitro results are consistent with our observation of increased circulating levels of isoforms of SAA in SJIA flare. A biomarker panel based on the unique protein derivatives we identified as optimal for SJIA will require generation of specific detection reagents.

We validated a subset of our DIGE results using ELISA as an independent method. ROC curve analysis suggests that the 7 ELISA panel may aid in diagnosis of SJIA, as it was better than CRP or S100A8/9 at classifying SJIA versus acute FI. However, an important caveat is that the SJIA F subjects studied with our panel were not all new onset, untreated cases, which would be the best comparator group. The ELISA panel also might be useful to distinguish SJIA flare from inter-current infection in a febrile child with known SJIA. A prospective study with SJIA subjects will be required to address this potential clinical utility. Nonetheless, our panel appears to provide stronger classifying power than any single biomarker alone.

Our data suggest that certain changes in plasma protein profiles occur in advance of clinically detectable disease activity. It has been reported that calprotectin levels rise in advance of clinical flare (Schulze et al. (2004) Clin. Exp. Rheumatol. 22:368-373). In unsupervised analysis of our DIGE data, one SJIA F sample clustered with the Q samples. This subject had active disease at the time of sample draw, but entered clinical quiescence over the next 2 months. Based on the DIGE analysis, SAA had already normalized in the flare sample from this subject, suggesting this protein changes earlier than others. APO A-1 spots were also similar to a quiescent pattern; this protein may contribute to resolution of a flare by inhibiting monocyte activation and synthesis of pro-inflammatory cytokines (Hyka et al. (2001) Blood 97:2381-2389). The 7-member ELISA panel also classified 4 out of 5 quiescent samples correctly as “pre-flare”.

In addition to the diagnostic challenges associated with fevers of unknown origin and fever in children with SJIA, prognostic challenges are prominent in SJIA. The clinical course is variable, ranging from a monocyclic episode with recovery in about 50% of subjects to a chronic, either polycyclic or persistent, condition, often with severe joint damage (Lomater et al. (2000) Journal of Rheumatology 27:491-496; Sandborg et al. (2006) Journal of Rheumatology 33:2322-2329). Only subsets of SJIA patients respond to currently available therapies (Wallace et al. (2005) Arthritis and Rheumatism 52:3554-3562; Gattorno et al. (2008) Arthritis and Rheumatism 58:1505-1515). Complications of SJIA include growth failure, macrophage activation syndrome and amyloidosis, the latter two being potentially life-threatening (Woo (2006) Nature Clinical Practice Rheumatology 2:28-34; Sawhney et al. (2001) Archives of Disease in Childhood 85:421-426). Proteomic strategies provide an attractive approach to discover prognostic biomarkers in SJIA. We have found a preliminary suggestion that our ELISA panel may identify those subjects at onset who will have a monocyclic course (JLP, unpublished data). In this regard, it is encouraging that a recent 2D-DIGE analysis of synovial fluid provided evidence for markers that predict the transition from oligoarticular to polyarticular disease in a subset of oligoarticular-onset JIA subjects (Gibson et al. (2009) J. Proteomics 72:656-676).

TABLE 1A
Demographics of SJIA and Poly JIA subjects for samples
analyzed by 2D-DIGE

| | SJIA F | SJIA Q | PolyJIA F | PolyJIA Q |
|---|---|---|---|---|
| N (# of subjects) | 10 | 10 | 5 | 5 |
| No. male/no. | 5/5 | 5/5 | 0/5 | 0/5 |
| female | | | | |
| Median age in | 8.5 | 10 | 18 | 19 |
| years | (2-16) | (2-17) | (10-18) | (12-21) |
| (range) | | | | |
| Caucasian | 4 | 4 | 0 | 0 |
| Hispanic | 3 | 3 | 4 | 4 |
| Asian | 3 | 3 | 0 | 0 |
| African | 0 | 0 | 1 | 1 |
| American | | | | |
| Median WBC | 15.5* | 10.2 | 8.8 | 7.6 |
| (×10³/μl) | (9.6-31.2) | (4.5-13.1) | (7.8-15.1) | (5.9-12.3) |
| (range) | | | | |
| Median platelets | 505* | 291 | 463 | 275 |
| (×10³/μl) | (384-786) | (219-536) | (211-497) | (180-404) |
| (range) | | | | |
| Median ESR | 86* | 9 | 35 | 11 |
| (mm/hr) | (42-143) | (0-15) | (16-78) | (6-30) |
| (range) | | | | |
| Median | 0.24 | 0 | 0 | 0 |
| prednisone | (0-5) | (0-0.28) | (0-0.1) | (0-0.03) |
| dose, | | | | |
| mg/kd/day | | | | |
| (range) | | | | |
| Methotrexate, # | 2 | 4 | 3 | 1 |
| subjects | | | | |
| Anti-TNF, # | 2 | 3 | 1 | 5 |
| subjects | | | | |

*Statistically significant difference(p < 0.05) from SJIA Q by student's t-test
F: Flare;
Q: Quiescent;
WBC: white blood count;
ESR: erythrocyte sedimentation rate

TABLE 1B
Demographics of SJIA F, KD, and FI subjects for samples analyzed by
2D-DIGE

| | SJIA F | KD | FI |
|---|---|---|---|
| N (# of subjects) | 10 | 12 | 12 |
| M/F | 5/5 | 9/3 | 6/6 |
| Median age in years | 8.5 (2-16) | 2 (0.3-9) | 4 (0.7-18) |
| (range) | | | |
| Caucasian | 4 | 4 | 5 |
| Hispanic | 3 | 5 | 4 |
| Asian | 3 | 2 | 1 |
| African American | 0 | 1 | 2 |
| Median WBC (×10³/μl) | 15.5 (9.6-31.2) | ND | ND |
| (range) | | | |
| Median platelets (×10³/μl) | 505 (384-786) | ND | ND |
| (range) | | | |
| Median ESR (mm/hr) | 86 (42-143) | ND | ND |
| (range) | | | |

F: Flare;
KD: Kawasaki Disease;
FI: Febrile illness;
WBC: white blood count;
ESR: Erythrocyte sedimentation rate;
ND: Not done in all subjects

TABLE 2A
Demographic characteristics of SJIA and Poly JIA subjects for ELISA
analyses

| | SJIA F | SJIA Q | Poly JIA F | Poly JIA Q |
|---|---|---|---|---|
| N (# of | 22 | 22 | 23 | 15 |
| subjects) | | | | |
| M/F | 13/9 | 12/10 | 3/20 | 2/13 |
| Median age | 9.5 | 11 | 15 | 14 |
| in years | (3-18) | (2-19) | (2-21) | (3-21) |
| (range) | | | | |
| Caucasian | 8 | 7 | 12 | 7 |
| Hispanic | 8 | 10 | 8 | 6 |
| Asian | 4 | 3 | 2 | 1 |
| African | 2 | 2 | 1 | 1 |
| American | | | | |
| Median | 12.6** | 6.8 | N/A | N/A |
| WBC | (7.7-34.2) | (3.6-12.4) | | |
| (×10³/μl) | | | | |
| (range) | | | | |
| Median | 475K** | 305K | N/A | N/A |
| platelets | (239K-766K) | (215K-429K) | | |
| (×10³/μl) | | | | |
| (range) | | | | |
| Median ESR | 55** | 10 | N/A | N/A |
| (mm/hr) | (5-105) | (0-38) | | |
| (range) | | | | |
| Median | 0.125** | 0 | 0 | 0 |
| prednisone | (1-1.35) | (0-0.28) | (0-0.12) | |
| dose, mg/ | | | | |
| kg/day | | | | |
| (range) | | | | |
| Methotrex- | 10 | 7 | 12 | 9 |
| ate, # | | | | |
| subjects | | | | |
| Anti-TNF, | 8 | 7 | 16 | 14 |
| # subjects | | | | |

**Statistically significant difference (p < 0.05) from SJIA Q by student's t-test
WBC: white blood count;
ESR: erythrocyte sedimentation rate;
N/A: not applicable

TABLE 2B
Demographics of SJIA F, KD, and FI subjects for samples
used in ELISA analyses

| | SJIA F | KD | FI |
|---|---|---|---|
| N (# of subjects) | 22 | 10 | 27 |
| M/F | 13/9 | 6/4 | 15/12 |
| Median age in years | 9.5* | 7.35 | 2.5 |
| (range) | (3-18) | (4-15) | (0.2-17.5) |
| Caucasian | 8 | 3 | 7 |
| Hispanic | 8 | 4 | 15 |
| Asian | 4 | 1 | 1 |
| African American | 2 | 2 | 4 |
| Median WBC | 12.5 | ND | ND |
| (×10³/μl) (range) | (7.7-34.2) | | |
| Median platelets | 475 | ND | ND |
| (×10³/μl) (range) | (239-766) | | |
| Median ESR (mm/hr) | 86 | ND | ND |
| (range) | (5-108) | | |

F: Flare;
KD: Kawasaki Disease;
FI: Febrile illness;
WBC: White blood count;
ESR: Erythrocyte sedimentation rate;
ND: not done in all subjects
*Statistically different from KD and FI by ANOVA

TABLE 3
Demographics of SJIA subjects for prediction of flare analysis

| | SJIA QQ | SJIA QF |
|---|---|---|
| N (# of subjects) | 10 | 5 |
| M/F | 6/4 | 3/2 |
| Median age in years | 10.5 | 11 |
| (range) | (5-17) | (6-13) |
| Caucasian | 4 | 2 |
| Hispanic | 3 | 3 |
| Asian | 2 | 0 |
| African American | 1 | 0 |
| Median WBC (×10³/μl) | 5.8 | 7.1 |
| (range) | (4.2-7.3) | (4.2-8.5) |
| Median platelets (×10³/μl) | 263 | 351 |
| (range) | (170-381) | (228-369) |
| Median ESR (mm/hr) | 8 | 10 |
| (range) | (3-16) | (4-23) |
| Median prednisone dose, | 0 | 0.5 |
| mg/kg/day (range) | (0-1) | (0-2) |
| Methotrexate, # subjects | 4 | 2 |
| Anti-TNF, # subjects | 6 | 1 |

QQ: Quiescence control;
QF: Quiescence preceding flare
WBC: white blood count;
ESR: erythrocyte sedimentation rate

SUPPLEMENTARY TABLE 1A
Systemic scores

| | Severity | |
|---|---|---|
| Score | level | Systemic symptoms (in past 2 weeks except as noted) |
| 0 | none | no active disease |
| 1 | mild | having any one of the following: (1) rash (2) rare |
| | | fevers <10 days in past month (3) ESR 40-90 (4) |
| | | platelets >450,000 <550,000 |
| 2 | moderate | having at least 3 of the following: |
| | | (1) rash, (2) fever >10 days in past month, (3) |
| | | WBC >20,000 (4) ESR >90, (5) platelet |
| | | >550,000 (6) d-dimers>250-500 |
| 3 | severe | having any one of the following symptoms*: |
| | | (1) pneumonitis (2) pericarditis (3) pleural |
| | | effusion (4) MAS |

*Pulmonary involvement: symptomatic pleuritis, pleural effusion, or pneumonitis confirmed by radiograph.
Cardiac involvement: symptomatic myocarditis or pericarditis confirmed by echocardiogram.
Macrophage activation syndrome (MAS):
(1) acute illness with fever, bruising, petechiae, or mucosal bleeding;
(2) hepatomegaly or splenomegaly;
(3) a drop in red cell blood count, white cell blood count, platelet count, or sedimentation rate;
(4) prolonged partial thromboplastin time or prothrombin time; and
(5) hypo-fibrinogenemia

SUPPLEMENTARY TABLE 1B
Arthritis scores (polyJIA)

| Score | Severity level | Arthritis |
|---|---|---|
| 0 | none | no active disease |
| 1 | mild | 1-10 active joints** |
| 2 | moderate | 10-20 active joints |
| 3 | severe | >20 active joints |

SUPPLEMENTARY TABLE 1C
Arthritis scores (SJIA)

| Score | Severity level | Arthritis |
|---|---|---|
| A | none | no joint involvement |
| B | mild | <5 active joints** |
| C | moderate | 5-10 active joints |
| D | severe | >10 active joints |

While the preferred embodiments of the invention have been illustrated and described, it will be appreciated that various changes can be made therein without departing from the spirit and scope of the invention.

What is claimed is:

1. A method for diagnosing systemic juvenile idiopathic arthritis (SJIA) in a subject, the method comprising: measuring the level of a plurality of biomarkers in a biological sample derived from the subject, wherein the plurality of biomarkers comprises alpha-2-macroglobulin (A2M), apolipoprotein A1 (APO-AI), C-reactive protein (CRP), haptoglobin (HP), calgranulin A (S100A8/MRP8), calgranulin B (S100A9/MRP14), serum amyloid A (SAA), and serum amyloid P (SAP); and analyzing the levels of the biomarkers in conjunction with respective reference value ranges for said plurality of biomarkers, wherein differential expression of one or more biomarkers in the biological sample compared to one or more biomarkers in a control sample from a normal subject indicates that the subject has SJIA.

2. The method of claim 1, further comprising determining whether the subject is in a state of SJIA disease flare or a state of SJIA disease quiescence.

3. The method of claim 1, further comprising distinguishing a diagnosis of SJIA from a diagnosis of infectious illness in the subject.

4. The method of claim 1, further comprising distinguishing a diagnosis of SJIA from a diagnosis of acute febrile illness in the subject.

5. The method of claim 1, further comprising distinguishing a diagnosis of SJIA from a diagnosis of Kawasaki disease in the subject.

6. The method of claim 1, further comprising distinguishing a diagnosis of SJIA from a diagnosis of another JIA disease subtype in the subject.

7. The method of claim 6, wherein the JIA subtype is polyarticular JIA.

8. The method of claim 1, wherein the plurality of biomarkers further comprises transthyretin (TTR) or calgranulin C (S100A12).

9. The method of claim 1, wherein the plurality of biomarkers further comprises one or more proteins selected from the group consisting of transthyretin (TTR), complement factor H (CFH), gelsolin (GSN), complement C4 (C4), alpha-1-acid glycoprotein (AGP1), alpha-1-antichymotrypsin (ACT), and apolipoprotein A-IV (APO A-IV).

10. The method of claim 1, wherein the plurality of biomarkers further comprises one or more proteins selected from the group consisting of alpha-1-antichymotrypsin (ACT), alpha-1-acid glycoprotein (AGP1), inter-alpha-trypsin inhibitor light chain (AMBP), apolipoprotein A-IV (APO A-IV), apolipoprotein D (APO D), apolipoprotein E (APO E), apolipoprotein L1 (APO L1), antithrombin III (ATIII), complement C3 (C3), complement C4 (C4), complement C9 (C9), fibrinogen β (FGB), fibrinogen γ (FGG), gelsolin (GSN), complement factor H-related protein 1 (H36), kininogenin (KLKB1), transthyretin (TTR), and vitamin D binding protein (VDB).

11. The method of claim 1, wherein the biological sample is plasma or blood.

12. The method of claim 1, wherein the subject is a human being.

13. The method of claim 1, wherein measuring the level of the plurality of biomarkers comprises performing an enzyme-linked immunosorbent assay (ELISA), a radioimmunoassay (RIA), an immunofluorescent assay (IFA), a sandwich assay, magnetic capture, microsphere capture, a Western Blot, surface enhanced Raman spectroscopy (SERS), flow cytometry, or mass spectrometry.

14. The method of claim 13, wherein measuring the level of a biomarker comprises contacting an antibody with the biomarker, wherein the antibody specifically binds to the biomarker, or a fragment thereof containing an antigenic determinant of the biomarker.

15. The method of claim 14, wherein the antibody is selected from the group consisting of a monoclonal antibody, a polyclonal antibody, a chimeric antibody, a recombinant fragment of an antibody, an Fab fragment, an Fab′ fragment, an F(ab)₂ fragment, an Fᵥ fragment, and an scFᵥ fragment.

16. A method for predicting an SJIA flare in a subject in advance of clinical symptoms of flare, the method comprising: measuring the level of a plurality of biomarkers in a biological sample derived from the subject, wherein the plurality of biomarkers comprises alpha-2-macroglobulin (A2M), apolipoprotein A1 (APO-AI), C-reactive protein (CRP), haptoglobin (HP), calgranulin A (S100A8/MRP8), calgranulin B (S100A9/MRP14), serum amyloid A (SAA), and serum amyloid P (SAP); and analyzing the levels of the biomarkers in conjunction with respective reference value ranges for said plurality of biomarkers, wherein differential expression of one or more biomarkers in the biological sample compared to one or more biomarkers in a control sample from a normal subject indicates that the subject will have the SJIA flare at some time in the next 9 weeks.

17. The method of claim 16, wherein the plurality of biomarkers further comprises transthyretin (TTR) or calgranulin C (S100A12).

18. The method of claim 16, wherein the plurality of biomarkers further comprises one or more proteins selected from the group consisting of transthyretin (TTR), complement factor H (CFH), gelsolin (GSN), complement C4 (C4), alpha-1-acid glycoprotein (AGP1), alpha-1-antichymotrypsin (ACT), and apolipoprotein A-IV (APO A-IV).

19. The method of claim 16, wherein the plurality of biomarkers further comprises one or more proteins selected from the group consisting of alpha-1-antichymotrypsin (ACT), alpha-1-acid glycoprotein (AGP1), inter-alpha-trypsin inhibitor light chain (AMBP), apolipoprotein A-IV (APO A-IV), apolipoprotein D (APO D), apolipoprotein E (APO E), apolipoprotein L1 (APO L1), antithrombin III (ATIII), complement C3 (C3), complement C4 (C4), complement C9 (C9), fibrinogen β (FGB), fibrinogen γ (FGG), gelsolin (GSN), complement factor H-related protein 1 (H36), kininogenin (KLKB1), transthyretin (TTR), and vitamin D binding protein (VDB).

20. A method for evaluating the effect of an agent for treating SJIA in a subject, the method comprising: analyzing the level of each of one or more biomarkers in samples derived from the subject before and after the subject is treated with said agent, in conjunction with respective reference value ranges for said one or more biomarkers, wherein the one or more biomarkers comprises A2M, APO-AI, CRP, HP, S100A8/S100A9, SAA, and SAP, or any combination thereof.

21. A method for monitoring the efficacy of a therapy for treating SJIA in a subject, the method comprising: analyzing the level of each of one or more biomarkers in samples derived from the subject before and after the subject undergoes said therapy, in conjunction with respective reference value ranges for said one or more biomarkers, wherein the one or more biomarkers comprises A2M, APO-AI, CRP, HP, S100A8/S100A9, SAA, and SAP, or any combination thereof.

22. A biomarker panel comprising A2M, APO-AI, CRP, HP, S100A8/S100A9, SAA, and SAP.

23. The biomarker panel of claim 22, wherein the biomarker panel consists of A2M, APO-AI, CRP, HP, S100A8/S100A9, SAA, and SAP.

24. The biomarker panel of claim 22, further comprising one or more biomarkers selected from the group consisting of transthyretin (TTR), calgranulin C (S100A12), complement factor H (CFH), gelsolin (GSN), complement C4 (C4), alpha-1-acid glycoprotein (AGP1), alpha-1-antichymotrypsin (ACT), and apolipoprotein A-IV (APO A-IV).

25. The biomarker panel of claim 22, further comprising one or more biomarkers selected from the group consisting of alpha-1-antichymotrypsin (ACT), alpha-1-acid glycoprotein (AGP1), inter-alpha-trypsin inhibitor light chain (AMBP), apolipoprotein A-IV (APO A-IV), apolipoprotein D (APO D), apolipoprotein E (APO E), apolipoprotein L1 (APO L1), antithrombin III (ATIII), complement C3 (C3), complement C4 (C4), complement C9 (C9), fibrinogen β (FGB), fibrinogen γ (FGG), gelsolin (GSN), complement factor H-related protein 1 (H36), kininogenin (KLKB1), transthyretin (TTR), and vitamin D binding protein (VDB).

26. A kit comprising agents for measuring the level of at least seven biomarkers of interest, wherein the at least seven biomarkers of interest comprise A2M, APO-AI, CRP, HP, S100A8/S100A9, SAA, and SAP.

27. The kit of claim 26, wherein the agents comprise at least one of an antibody that specifically binds to A2M, an antibody that specifically binds to APO-AI, an antibody that specifically binds to CRP, an antibody that specifically binds to HP, an antibody that specifically binds to S100A8/S100A9, an antibody that specifically binds to SAA, and an antibody that specifically binds to SAP.

28. The kit of claim 26, further comprising one or more control reference samples.

29. The kit of claim 26, further comprising information, in electronic or paper form, comprising instructions to correlate the detected levels of each of the at least seven biomarkers of interest with SJIA.

30. The kit of claim 26, further comprising at least seven separate containers inside a package, wherein each separate container contains a respective one of the agents.

31. The kit of claim 26, further comprising reagents for performing an immunoassay.

32. The kit of claim 31, wherein the immunoassay is an ELISA.
